(19) United States
(12) Patent Application Publication
(10) Pub. No.: US 2022/0152024 A1
(43) Pub. Date: May 19, 2022

(54) COMBINATIONS USEFUL IN A METHOD FOR TREATING SARCOMA

(71) Applicant: PTC THERAPEUTICS, INC., South Plainfield, NJ (US)

(72) Inventors: Marla L. Weetall, Morristown, NJ (US); Liangxian Cao, East Brunswick, NJ (US); Josephine Sheedy, Bordentown, NJ (US); Robert Spiegel, Westfield, NJ (US)

(21) Appl. No.: 17/441,355

(22) PCT Filed: Mar. 27, 2020
(86) PCT No.: PCT/US2020/025532

§ 371 Date: Sep. 21, 2021

Related U.S. Application Data
(60) Provisional application No. 62/825,017, filed on Mar. 27, 2019.

(51) Int. Cl. A61K 31/506; A61K 45/06; A61P 35/00

(52) U.S. Cl. CPC A61K 31/506; A61P 35/00; A61K 45/06

(57) ABSTRACT

One aspect described herein includes a method for treating sarcoma in a subject in need thereof comprising, administering to the subject an effective amount of a small molecule compound. More particularly, another aspect described herein includes a method for treating sarcoma in a subject in need thereof comprising, administering to the subject an effective amount of the small molecule compound described herein in combination with a chemotherapeutic agent.

### CROSS-REFERENCE TO RELATED APPLICATIONS

This application claims priority to U.S. Provisional Application No. 62/825,017, filed on Mar. 27, 2019, the contents of which are incorporated by reference herein.

### FIELD

Described herein is a method for treating sarcoma in a subject in need thereof comprising, administering to the subject an effective amount of a small molecule compound. More particularly described herein is a method for treating sarcoma in a subject in need thereof comprising, administering to the subject an effective amount of a small molecule compound alone or in combination with a chemotherapeutic agent.

### BACKGROUND

Soft tissue sarcoma (STS) is a cancer derived from mesenchymal tissues, such as smooth muscle (uterus, stomach, small intestine, retroperitoneum, blood vessels and skin), tendons, fat, lymph vessels, vascular tissue and nerves, as well as cartilage and other tissues around joints. Although only about 1% of total new cancers annually, there are approximately 15,000 new cases of sarcoma diagnosed each year in the United States. Smooth muscle sarcomas are more common, constituting about 5-10% of all soft tissue sarcomas. In one such example of sarcoma, the aggressive, locally recurrent, unresectable and metastatic relapsed/refractory leiomyosarcoma (LMS) represents about 24% of those sarcomas.

The standard of care (SOC) for treating sarcoma includes surgery, radiation and chemotherapy. However, there has been little progress in the treatment of aggressive LMSs. First line treatments include the combinations of Adriamycin® or Rubex® (doxorubicin), with or without Ifex® (ifosfamide), and Gemzar® (gemcitabine) with Taxotere® (docetaxel). The cytotoxic agent trabectedin and the angiogenic receptor tyrosine kinase inhibitor pazopanib have been recently approved for treatment of LMS in a second-line or later-line setting, providing a progression-free survival (PFS) benefit of several months, albeit with no demonstrated improvement in overall survival (OS) (van der Graaf 2012, Demetri 2016). DTIC-Dome® (dacarbazine), widely utilized for the treatment of soft tissue sarcoma, is often used in the later-line setting and achieves response rates of approximately 10% for LMS.

Based upon modest improvements for short-term response rate with the combination of trabectedin and pazopanib, dacarbazine remains a relevant choice for LMS therapy despite the low response rate. Accordingly, LMS represents a relatively common sarcoma subtype in need of new clinically effective chemotherapeutic agents or combinations thereof.

### SUMMARY

One aspect described herein is a method for treating sarcoma in a subject in need thereof comprising, administering to the subject an effective amount of 5-fluoro-2-(6-fluoro-2-methyl-1H-benzo[d]imidazol-1-yl)-N⁴-[4-(trifluoromethyl)phenyl]pyrimidine-4,6-diamine, having the structure of Compound 1:

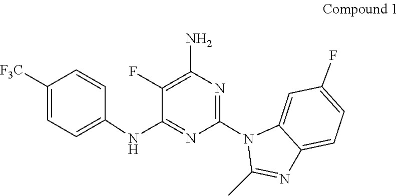 [SMILES: Cc1nc2ccc(F)cc2n1-c1nc(N)c(F)c(Nc2ccc(C(F)(F)F)cc2)n1]

or a pharmaceutically acceptable salt or pharmaceutical composition thereof.

Another aspect described herein is a method for treating sarcoma in a subject in need thereof comprising, administering to the subject an effective amount of Compound 1 or a pharmaceutically acceptable salt or pharmaceutical composition thereof in combination with an effective amount of at least one chemotherapeutic agent.

One aspect described herein is a use of Compound 1 or a pharmaceutically acceptable salt or pharmaceutical composition thereof for treating sarcoma in a subject in need thereof comprising, administering to the subject an effective amount of Compound 1 or a pharmaceutically acceptable salt or pharmaceutical composition thereof.

Another aspect described herein is a use of Compound 1 or a pharmaceutically acceptable salt or pharmaceutical composition thereof for treating sarcoma in a subject in need thereof comprising, administering to the subject an effective amount of Compound 1 or a pharmaceutically acceptable salt or pharmaceutical composition thereof in combination with an effective amount of at least one chemotherapeutic agent.

One aspect described herein is a use of Compound 1 or a pharmaceutically acceptable salt or pharmaceutical composition thereof for preparing a medicament for treating sarcoma in a subject in need thereof comprising, administering to the subject an effective amount of the medicament.

Another aspect described herein is a use of Compound 1 in preparing a medicament for use in treating sarcoma in a subject in need thereof comprising, administering to the subject an effective amount of the medicament in combination with an effective amount of at least one chemotherapeutic agent.

In one aspect, chemotherapeutic combination therapies include administration of Compound 1 in combination with at least one chemotherapeutic agent, wherein the at least one chemotherapeutic agent is selected from the group consisting of DTIC-Dome® (dacarbazine), Taxotere® (docetaxel), Adriamycin® or Rubex® (doxorubicin), Doxil® (liposomal doxorubicin), gemcitabine, epirubicin, eribulin, ifosfamide, temozolomide, trabectedin and Oncovin® (vincristine).

In another aspect, chemotherapeutic combination therapies include administration of Compound 1 in combination with at least one chemotherapeutic agent, wherein the at least one chemotherapeutic agent is selected from the group consisting of dacarbazine, docetaxel, doxorubicin, liposomal doxorubicin, gemcitabine, epirubicin, eribulin, ifosfamide, temozolomide, trabectedin, and vincristine.

In another aspect, chemotherapeutic combination therapies include administration of Compound 1 in combination with at least one chemotherapeutic agent, wherein the at least one chemotherapeutic agent is selected from the group consisting of dacarbazine, docetaxel, doxorubicin, liposomal doxorubicin, gemcitabine, and vincristine.

In another aspect, chemotherapeutic combination therapies include administration of Compound 1 in combination with at least one chemotherapeutic agent, wherein the at least one chemotherapeutic agent is selected from the group consisting of dacarbazine, docetaxel, doxorubicin, liposomal doxorubicin, and vincristine.

In another aspect the chemotherapeutic combination therapies include administration of Compound 1 in combination with at least one chemotherapeutic agent wherein the at least one chemotherapeutic agent is dacarbazine.

In another aspect the chemotherapeutic combination therapies include administration of Compound 1 in combination with at least one chemotherapeutic agent wherein the at least one chemotherapeutic agent is docetaxel.

In another aspect the chemotherapeutic combination therapies include administration of Compound 1 in combination with at least one chemotherapeutic agent wherein the at least one chemotherapeutic agent is doxorubicin.

In another aspect the chemotherapeutic combination therapies include administration of Compound 1 in combination with at least one chemotherapeutic agent wherein the at least one chemotherapeutic agent is liposomal doxorubicin.

In another aspect the chemotherapeutic combination therapies include administration of Compound 1 in combination with at least one chemotherapeutic agent wherein the at least one chemotherapeutic agent is vincristine.

### BRIEF DESCRIPTION OF THE DRAWINGS

FIG. 1A and FIG. 1B each show a combination of Compound 1 and dacarbazine (DTIC), resulting in a synergistic reduction in mean tumor volume in an SK-LMS-1 mouse model after treatment with the combination in comparison to Compound 1 alone, DTIC alone and vehicle; where biw represents dose administration two times per week, tiw represents dose administration three times per week and, where qd5 represents dose administration once per day for five days. Comparison of FIG. 1A and FIG. 1B show a synergistic dose response reduction in mean tumor volume after treatment with a combination of Compound 1 (12.5 mg/kg PO biw) and DTIC (4 mg/kg IP tiw) (see, FIG. 1A) compared to treatment with a combination of Compound 1 (12.5 mg/kg PO biw) and DTIC (21 mg/kg IP qd5) (see, FIG. 1B).

FIG. 2A and FIG. 2B each show a combination of Compound 1 and docetaxel, resulting in a synergistic reduction in mean tumor volume in an SK-UT-1 leiomyosarcoma (LMS) mouse model after treatment with the combination in comparison to Compound 1 alone, docetaxel alone and vehicle; where biw represents dose administration two times per week and, where qw6 represents dose administration once per week for six weeks. Comparison of FIG. 2A and FIG. 2B show a dose dependent suppression of mean tumor growth after treatment with a combination of Compound 1 (12.5 mg/kg PO biw) and docetaxel (5 mg/kg IP biw for six doses) (see, FIG. 2A) compared to treatment with a combination of Compound 1 (12.5 mg/kg PO biw) and docetaxel (15 mg/kg IP qw6) (see, FIG. 2B).

FIG. 3A and FIG. 3B each show a combination of Compound 1 and doxil, resulting in a synergistic reduction in mean tumor volume in an SK-LMS-1 mouse model after treatment with the combination in comparison to Compound 1 alone, doxil alone and vehicle; where biw represents dose administration two times per week and, where qw5 represents dose administration once per week for five weeks. Comparison of FIG. 3A and FIG. 3B show a dose dependent suppression of mean tumor growth after treatment with a combination of Compound 1 (12.5 mg/kg PO biw) and doxil (3 mg/kg IP qw5) (see, FIG. 3A) compared to treatment with a combination of Compound 1 (12.5 mg/kg PO biw) and doxil (9 mg/kg IP qw5) (see, FIG. 3B).

FIG. 4 shows a combination of Compound 1 and doxorubucin, resulting in a synergistic reduction in mean tumor volume in a HT1080 fibrosarcoma mouse model after treatment with the combination in comparison to Compound 1 alone (15 mg/kg PO biw), doxorubucin alone (0.3 mg/kg IP q2d) and vehicle; where biw represents dose administration two times per week and, where q2d represents dose administration once per day every two days.

FIG. 5 summarizes the duration of Compound 1 treatment alone in an all-solid tumor (AST) Phase 1a clinical trial (NCT02404480) of 31 evaluable patients to determine safety and pharmacokinetics. The initial human dose of 0.65 mg/kg was based on a rat model MTD of 40 mg/kg, with subsequent higher human dose ranging at 1.3 mg/kg, 2.6 mg/kg, 5.2 mg/kg, 7 mg/kg, and 10 mg/kg. The Standard 3+3 trial design, with 3 patients per cohort, allowed dose escalation for each cohort absent a dose-limiting toxicity (DLT), enabling the next cohort to be treated at the next higher dose. In the event any one patient in the cohort experienced a DLT at a particular dose, the next cohort would be treated at the same dose. A full pharmacokinetic review of each patient was taken on Day 1 and Day 29, with a partial review taken on Day 15.

FIGS. 6A-D summarize the pharmacokinetics of Compound 1 administered as a monotherapy in humans (as measured by AUC_{Last} and Cₘₐₓ) two times per week in the AST trial, wherein the dotted line in each figure indicates free drug concentration. FIGS. 6A and 6B show an estimated target AUC_{Last} of about 13,125 hr-ng/mL (FIG. 6A) and an estimated target Cₘₐₓ of about 688 ng/mL (FIG. 6B), correlating to a dose in a human patient of about 2.0 mg/kg or higher or about 1.4 mg/kg or higher of Compound 1, respectively, required to maintain the human AUC and Cₘₐₓ, respectively, above the target concentrations predicted to be effective.

FIGS. 6C and 6D show an estimated target AUC_{Last} of 15,625 hr-ng/mL (FIG. 6C) and an estimated target Cₘₐₓ of about 859 ng/mL (FIG. 6D), correlating to a dose in a human patient of about 2.3 mg/kg or higher or about 1.8 mg/kg or higher of Compound 1, respectively, required to maintain the human AUC and Cₘₐₓ above the target concentrations predicted to be effective.

FIG. 7A and FIG. 7B each show a combination of Compound 1 and vincristine, resulting in a reduction in mean tumor volume in an HT1080 fibrosarcoma xenograft mouse model after treatment with the combination in comparison to Compound 1 alone, vincristine alone and vehicle; where biw represents dose administration two times per week and, where tiw represents dose administration three times per week. Comparison of FIG. 7A and FIG. 7B show for each agent a dose dependent decrease in mean tumor growth after treatment with a combination of Compound 1 (12.5 mg/kg PO biw) and vincristine (0.1 mg/kg IP tiw) (see, FIG. 7A) compared to treatment with a combination of Compound 1 (12.5 mg/kg PO biw) and vincristine (0.3 mg/kg IP tiw) (see, FIG. 7B).

### DETAILED DESCRIPTION

One aspect described herein is a method for treating sarcoma in a subject in need thereof comprising, administering to the subject an effective amount of 5-fluoro-2-(6-fluoro-2-methyl-1H-benzo[d]imidazol-1-yl)-N⁴-[4-(trifluoromethyl)phenyl]pyrimidine-4,6-diamine, having the structure of Compound 1:

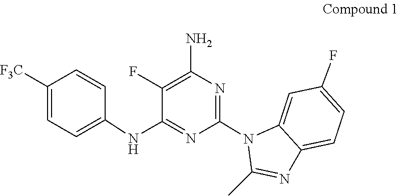 [SMILES: Cc1nc2ccc(F)cc2n1-c1nc(N)c(F)c(Nc2ccc(C(F)(F)F)cc2)n1]

or a pharmaceutically acceptable salt or pharmaceutical composition thereof.

Another aspect described herein is a method for treating sarcoma in a subject in need thereof comprising, administering to the subject an effective amount of Compound 1 or a pharmaceutically acceptable salt or pharmaceutical composition thereof in combination with an effective amount of at least one chemotherapeutic agent.

One aspect described herein is a use of Compound 1 or a pharmaceutically acceptable salt or pharmaceutical composition thereof for treating sarcoma in a subject in need thereof comprising, administering to the subject an effective amount of Compound 1 or a pharmaceutically acceptable salt or pharmaceutical composition thereof.

Another aspect described herein is a use of Compound 1 or a pharmaceutically acceptable salt or pharmaceutical composition thereof for treating sarcoma in a subject in need thereof comprising, administering to the subject an effective amount of Compound 1 or a pharmaceutically acceptable salt or pharmaceutical composition thereof in combination with an effective amount of at least one chemotherapeutic agent.

One aspect described herein is a use of Compound 1 or a pharmaceutically acceptable salt or pharmaceutical composition thereof for preparing a medicament for treating sarcoma in a subject in need thereof comprising, administering to the subject an effective amount of the medicament.

Another aspect described herein is a use of Compound 1 in preparing a medicament for use in treating sarcoma in a subject in need thereof comprising, administering to the subject an effective amount of the medicament in combination with an effective amount of at least one chemotherapeutic agent.

In one aspect, chemotherapeutic combination therapies include administration of Compound 1 in combination with at least one chemotherapeutic agent, wherein the at least one chemotherapeutic agent is selected from the group consisting of DTIC-Dome® (dacarbazine), Taxotere® (docetaxel), Adriamycin® or Rubex® (doxorubicin), Doxil® (liposomal doxorubicin), gemcitabine, epirubicin, eribulin, ifosfamide, temozolomide, trabectedin, and Oncovin® (vincristine).

In another aspect, chemotherapeutic combination therapies include administration of Compound 1 in combination with at least one chemotherapeutic agent, wherein the at least one chemotherapeutic agent is selected from the group consisting of dacarbazine, docetaxel, doxorubicin, liposomal doxorubicin, gemcitabine, epirubicin, eribulin, ifosfamide, temozolomide, trabectedin, and vincristine.

In another aspect, chemotherapeutic combination therapies include administration of Compound 1 in combination with at least one chemotherapeutic agent, wherein the at least one chemotherapeutic agent is selected from the group consisting of dacarbazine, docetaxel, doxorubicin, liposomal doxorubicin, gemcitabine, and vincristine.

In another aspect, chemotherapeutic combination therapies include administration of Compound al in combination with at least one chemotherapeutic agent, wherein the at least one chemotherapeutic agent is selected from the group consisting of dacarbazine, docetaxel, doxorubicin, liposomal doxorubicin, and vincristine.

Compound 1 and a method for making the same are disclosed in International Publication Number WO2014/081906 (cited as Compound 109).

### Definitions

As used herein, the term “about” means a range around a given value wherein the resulting value is substantially the same as the expressly recited value. In one aspect, “about” means within 25% of a given value or range. For example, the phrase “about 70% by weight” comprises at least all values from 52% to 88% by weight. In another aspect, the term “about” means within 10% of a given value or range. For example, the phrase “about 70% by weight” comprises at least all values from 63% to 77% by weight. In another aspect, the term “about” means within 7% of a given value or range. For example, the phrase “about 70% by weight” comprises at least all values from 65% to 75% by weight. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range was explicitly recited.

As used herein, the terms “therapies” and “therapy” can refer to any protocol(s), method(s), compositions, formulations, and/or agent(s) that can be used in the prevention, treatment, management, or amelioration of a condition or disorder or one or more symptoms thereof (e.g., sarcoma or one or more symptoms or one or more conditions associated therewith).

In one aspect, the terms “therapies” and “therapy” and “standard therapy” refer to one or more therapies, including a drug therapy such as chemotherapy, or adjuvant therapy, radiation, surgery, biological therapy, immunotherapy, supportive therapy, antiviral therapy and/or other therapies useful in treatment, management, prevention, or amelioration of a condition or disorder or one or more symptoms thereof (e.g., sarcoma or one or more symptoms or one or more conditions associated therewith).

In another aspect, the term chemotherapeutic agent refers to drugs used in chemotherapy to directly or indirectly inhibit the proliferation of rapidly growing cells, typically in the context of malignancy. Classified according to their mechanism of action, such drugs include alkylating agents, antimetabolites, topoisomerase inhibitors, mitotic inhibitors, those that directly or indirectly affect RNA or DNA related mechanisms, and the like.

In another aspect, the term “adjuvant therapy” refers to a therapy other than the use of Compound 1 or a pharmaceutically acceptable salt or pharmaceutical composition thereof as a monotherapy or a chemotherapeutic combination therapy, including administering Compound 1 in combination with at least one chemotherapeutic agent.

As used herein, the term “subject” may be used interchangeably with the term “patient,” wherein either or both terms refer to an individual in need thereof being administered a therapy that provides a beneficial or therapeutic effect, as described herein. In a specific aspect, the individual is a human.

As used herein, the term “effective amount” in the context of administering Compound 1 to a subject having sarcoma refers to the dose of Compound 1 that results in a beneficial or therapeutic effect. In one aspect, an “effective amount” of Compound 1 refers to an amount of Compound 1 which is sufficient to achieve at least one, two, three, four or more of the following beneficial or therapeutic effects: (i) inhibition of sarcoma; (ii) regression of the sarcoma; (iii) eradication, removal, or complete remission of the sarcoma; (iv) prevention of the development or onset of one or more symptoms associated with the sarcoma; (v) reduction or amelioration of the severity of one or more symptoms associated with the sarcoma; (vi) the reduction in the number of one or more symptoms associated with the sarcoma; (vii) amelioration of the severity of one or more symptoms associated with the sarcoma; (viii) reduction in the duration of one or more symptoms associated with the sarcoma; (ix) prevention in the recurrence of proliferation or one or more symptoms associated with the sarcoma; (x) a reduction in mortality; (xi) an increase in survival rate of subjects; (xii) an increase in relapse free survival; (xiii) an increase in the number of sarcoma subjects in remission; (xiv) reduction in hospitalization of a subject; (xv) reduction in hospitalization length; (xvi) a decrease in hospitalization rate; (xvii) an increase in the survival of a subject; (xviii) an increase in symptom-free survival of sarcoma subject; (xix) an increase in the length of a period of remission of sarcoma in a subject; (xx) improvement in quality of life (QOL) as assessed by methods well known in the art, e.g., QOL questionnaires and the like; (xxi) a reduction in proliferation from administration of Compound 1 before treatment with another chemotherapeutic agent; (xxii) a reduction in proliferation from administration of Compound 1 after treatment with another chemotherapeutic agent; (xxiii) a reduction in proliferation in a combination therapy from administration of Compound 1 with another chemotherapeutic agent; (xxiv) an additive antiproliferative effect in a combination therapy from administration of Compound 1 with another chemotherapeutic agent; (xxv) a synergistic antiproliferative effect in a combination therapy from administration of Compound 1 with another chemotherapeutic agent; (xxvi) a reduction in proliferation from administration of Compound 1 before therapy with radiation; (xxvii) a reduction in proliferation from administration of Compound 1 after therapy with radiation; (xxviii) a reduction in proliferation from administration of Compound 1 in a combination therapy with radiation; (xxix) a reduction in proliferation from administration of Compound 1 before treatment with surgery; (xxx) a reduction in proliferation from administration of Compound 1 in a combination treatment with surgery; (xxxi) enhancement of or improvement of the therapeutic effect from administration of Compound 1 with a palliative therapy; (xxxii) a decrease in the plasma concentration of BMI-1 in a subject having sarcoma; (xxxiii) a decrease in circulating proliferative cells in the plasma of a subject having sarcoma; (xxxiv) an alteration (e.g., a decrease or increase) in the plasma concentration of sarcoma biomarker in a subject having sarcoma (e.g., BMI-1, tubulin polymerization, apoptotic markers or tissue and the like); (xxxv) reduction in the concentration of BMI-1 in a biological specimen (e.g., plasma, serum, urine, or any other biofluids) from a subject having sarcoma; (xxxvi) proliferative cell count is reduced after administration of a therapy as described herein as measured by conventional methods available to one skilled in the art, such as magnetic resonance imaging (MRI), dynamic contrast-enhanced MRI (DCE-MRI), X-ray, computed tomography (CT) scan, positron emission tomography (PET) scan, 7-AAD fluorescence, or DAPI fluorescence; (xxxvii) proliferative cell count is maintained after administration of a therapy as described herein as measured by conventional methods available to one skilled in the art, such as magnetic resonance imaging (MRI), dynamic contrast-enhanced MRI (DCE-MRI), X-ray, computed tomography (CT) scan, positron emission tomography (PET) scan, 7-AAD fluorescence, or DAPI fluorescence; or, (xxxviii) proliferative cell count does not increase or increases by less than expected after administration of a therapy as described herein as measured by conventional methods available to one skilled in the art, such as magnetic resonance imaging (MRI), dynamic contrast-enhanced MRI (DCE-MRI), X-ray, computed tomography (CT) scan, or a positron emission tomography (PET) scan, 7-AAD fluorescence, or DAPI fluorescence.

As used herein, the term “in a 24 hour period” refers to a period of time over which a condition is maintained; for example, the effective amount of Compound 1 is identified when the mean plasma concentration of Compound 1 is achieved and maintained for a plurality of 24 hour periods. In other words, the mean plasma concentration of Compound 1 may be reached in a suitable time, which may be more or less than 24 hours.

As used herein, the term “a therapy as described herein” refers to a method of use for Compound 1 or a pharmaceutically acceptable salt or pharmaceutical composition thereof for use in treating or ameliorating sarcoma in a subject in need thereof comprising, administering to the subject an effective amount of Compound 1.

In one aspect of the therapy described herein, the use or method of use of Compound 1 includes a pharmaceutically acceptable salt or pharmaceutical composition thereof. In another aspect of the therapy described herein, the use or method of use of Compound 1 includes the use or method of use of Compound 1, a pharmaceutically acceptable salt or pharmaceutical composition of Compound 1, or a combination of Compound 1 or a pharmaceutically acceptable salt or pharmaceutical composition thereof with another chemotherapeutic agent(s), wherein the combination has synergistic antiproliferative activity. In another aspect, the other chemotherapeutic agent inhibits tubulin polymerization. In another aspect, the other chemotherapeutic agent inhibits BMI-1 functional activity.

As used herein, the term “pharmaceutically acceptable salt(s)” refers to a salt prepared from a pharmaceutically acceptable non-toxic acid or base including an inorganic acid and base and an organic acid and base; see, for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “Compound 1” refers to 5-fluoro-2-(6-fluoro-2-methyl-1H-benzo[d]imidazol-1-yl)-N⁴-[4-(trifluoromethyl)phenyl]pyrimidine-4,6-diamine or a pharmaceutically acceptable salt or pharmaceutical composition thereof. In various aspects, the term “Compound 1” refers to Compound 109 disclosed in International Publication No. WO2014/081906, which is incorporated in its entirety by reference herein.

### METHOD OF USE

Without being limited by theory, mechanistic studies have demonstrated that Compound 1 inhibits microtubule polymerization, binding to tubulin to cause a G2/M arrest, resulting in multiple cellular effects, including effects on mitosis, cell cycling and apoptosis.

In one aspect, Compound 1 may be administered as a monotherapy at doses that result in therapeutically effective target plasma concentrations.

In another aspect, Compound 1 may be administered as a combination therapy with at least one other chemotherapeutic agent at doses that result in therapeutically effective additive or synergistic plasma concentrations. When used in combination, the use of Compound 1 significantly enhances the activity of standard chemotherapeutics including dacarbazine, docetaxel, doxorubicin, liposomal doxorubicin, gemcitabine, vincristine and other tubulin binding agents.

Based on these data and previous preclinical studies, Cpd 1 is being evaluated in certain clinical studies, as posted on Clinical Trials.gov, including:

NCT02404480: Phase 1 Study, open-label, first-in-human, evaluated safety and pharmacokinetic (PK) profile for use of Compound 1 in patients with advanced solid tumor (AST) cancers, determined the RP2D (recommended phase II clinical trial dose) according to escalating dose levels.

NCT03206645: Phase 1b Study to evaluate safety and efficacy for treatment of ovarian cancer using Compound 1 in combination with standard paclitaxel and carboplatin for women with stage III or IV epithelial ovarian, primary peritoneal or fallopian tube cancer receiving neoadjuvant chemotherapy.

NCT03605550: Phase 1b Study to evaluate safety and efficacy for treatment of children with newly diagnosed Diffuse Intrinsic Pontine Glioma (DIPG) and/or High Grade Glioma (HGG) using Compound 1 in combination with radiation and chemotherapy. Patients diagnosed with DIPG/HGG may be treated with either radiation or surgery. The study will determine the dose of Compound 1 that can be given with radiation without causing serious side effects, the plasma and tumor tissue concentrations of Compound 1 prior to and/or during a surgery for removal of a recurrent tumor, and potential changes to tumor biology as a result of treatment with Compound 1.

NCT03761095: Phase 1b Study to evaluate safety and efficacy and determine the MTD (maximum tolerated dose) for treating patients having an advanced leiomyosarcoma using a combination of Compound 1 (200 mg PO biw) and dacarbazine (DTIC) (1000 mg/m² IV tiw).

As demonstrated herein, Compound 1 or a pharmaceutically acceptable salt or pharmaceutical composition thereof is a small molecule inhibitor of tubulin polymerization for use in treating or ameliorating sarcoma in a subject in need thereof comprising, administering to the subject an effective amount of Compound 1 or a pharmaceutically acceptable salt or pharmaceutical composition thereof.

In one aspect of the use or method of use described herein, the use or method of use of Compound 1 includes a pharmaceutically acceptable salt or pharmaceutical composition thereof.

In another aspect of the use or method of use described herein, the use or method of use of Compound 1 includes the use or method of use of Compound 1, the use or method of use of a pharmaceutically acceptable salt or pharmaceutical composition of Compound 1, or the use or method of use of a combination of Compound 1 or a pharmaceutically acceptable salt or pharmaceutical composition thereof with another chemotherapeutic agent(s), wherein the combination has additive or synergistic antiproliferative activity.

In another aspect, the other chemotherapeutic agent inhibits tubulin polymerization. In another aspect, the other chemotherapeutic agent affects DNA or DNA repair by various mechanisms.

In one aspect, the combinations described herein inhibit or reduce tubulin polymerization, which may also induce cell-cycle arrest in a proliferating cell or cell line are described herein.

In another aspect, a method for inhibiting or reducing tubulin polymerization to induce cell-cycle arrest in a proliferating cell or cell line comprises, exposure to Compound 1 or a pharmaceutically acceptable salt or pharmaceutical composition thereof with a proliferating cell or cell line. In another aspect, the proliferating cell or cell line may be naïve to treatment with a tubulin inhibitor or may be known to be affected by the inhibition or a reduction of tubulin polymerization.

In another aspect, non-limiting examples of such cells or cell lines are selected from HL-60, HeLa, HT1080, HCT116, HEK293, NCI H460, U-87MG, ASPC-1, PL-45, HPAF-2, PC-3, MDA-MB-231, MDA-MB-468, A431, SNU-1, AGS, Kato III, A549, Calu-6, A375, SYSY, SKOV3, Capan-1, sNF96.2, TIVE-L1, TIVE-L2, LNCaP cells and the like. In a more specific aspect, the cell or cell line may be sarcoma cell.

In one aspect, a method for inhibiting or reducing tubulin polymerization in a subject having sarcoma in need thereof comprises, administering an effective amount of Compound 1 or a pharmaceutically acceptable salt or pharmaceutical composition thereof to the subject as described herein.

In one aspect described herein, sarcoma that can be treated with the intended use described herein includes, and is not limited to, bone and connective tissue sarcomas selected from the group consisting of osteogenic sarcoma, bone sarcoma, osteosarcoma, chondrosarcoma, chordoma, synovioma, sarcomatous mesothelioma, Ewing's tumor, fibrosarcoma of bone, periosteal sarcoma, soft-tissue sarcomas, angiosarcoma, hemangiosarcoma, fibrosarcoma, Kaposi's sarcoma, leiomyosarcoma, liposarcoma, lymphangiosarcoma, rhabdomyosarcoma, synovial sarcoma, myxosarcoma, endotheliosarcoma, and lymphangioendotheliosarcoma.

In another aspect, the subject is diagnosed with sarcoma, wherein the sarcoma is selected from the group consisting of Ewing's tumor, fibrosarcoma, leiomyosarcoma, liposarcoma, and osteosarcoma.

In a specific aspect, the subject diagnosed with sarcoma is capable of being treated by a chemotherapeutic agent for inhibiting or reducing tubulin polymerization.

In a specific aspect, the subject diagnosed with sarcoma is capable of being treated by a chemotherapeutic agent for inhibiting or reducing BMI-1 function.

In a specific aspect, a method for inhibiting or reducing tubulin polymerization as described herein inhibits or reduces tubulin polymerization by about 5%, 10%, 15%, 20%, 25%, 30%, 35%, 40%, 45%, 50%, 55%, 60%, 65%, 80%, 85%, 90%, 95%, or 100% relative to tubulin polymerization prior to administration of Compound 1 to the subject, as assessed by methods well known in the art.

In a specific aspect, a method for inhibiting or reducing BMI-1 function as described herein inhibits BMI-1 function by about 5%, 10%, 15%, 20%, 25%, 30%, 35%, 40%, 45%, 50%, 55%, 60%, 65%, 80%, 85%, 90%, 95%, or 100% relative to BMI-1 function prior to administration of Compound 1 to the subject, as assessed by methods well known in the art.

In a specific aspect, a method for inhibiting or reducing tubulin polymerization as described herein inhibits or reduces tubulin polymerization in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to tubulin polymerization prior to administration of Compound 1 to the subject, as assessed by methods well known in the art.

In a specific aspect, a method for inhibiting or reducing BMI-1 function as described herein inhibits or reduces BMI-1 function in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to BMI-1 function prior to administration of Compound 1 to the subject, as assessed by methods well known in the art.

In a specific aspect, a method for inhibiting or reducing tubulin polymerization as described herein inhibits proliferation or reduces an in vitro or in vivo proliferating cell or cell line population by about 5%, 10%, 15%, 20%, 25%, 30%, 35%, 40%, 45%, 50%, 55%, 60%, 65%, 80%, 85%, 90%, 95%, or 100%, relative to the in vitro or in vivo proliferating cell or cell line population prior to administration of Compound 1 to the subject, as assessed by methods well known in the art.

In a specific aspect, a method for inhibiting or reducing BMI-1 function as described herein inhibits proliferation or reduces an in vitro or in vivo proliferating cell or cell line population by about 5%, 10%, 15%, 20%, 25%, 30%, 35%, 40%, 45%, 50%, 55%, 60%, 65%, 80%, 85%, 90%, 95%, or 100%, relative to the in vitro or in vivo proliferating cell or cell line population prior to administration of Compound 1 to the subject, as assessed by methods well known in the art.

In a specific aspect, a method for inhibiting or reducing tubulin polymerization as described herein inhibits proliferation or reduces an in vitro or in vivo proliferating cell or cell line population in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to the in vitro or in vivo proliferating cell or cell line population prior to administration of Compound 1 to the subject, as assessed by methods well known in the art.

In a specific aspect, a method for inhibiting or reducing BMI-1 function as described herein inhibits proliferation or reduces an in vitro or in vivo proliferating cell or cell line population in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to the in vitro or in vivo proliferating cell or cell line population prior to administration of Compound 1 to the subject, as assessed by methods well known in the art.

In various aspects, a method for inhibiting or reducing tubulin polymerization as described herein reduces the expression of GTP-bound αβ-tubulin subunits available for microtubule assembly in a subject as assessed by methods well known in the art, e.g., ELISA.

In various aspects, a method for inhibiting or reducing BMI-1 function as described herein reduces the plasma concentration of BMI-1 in a subject as assessed by methods well known in the art, e.g., ELISA.

In one aspect, a method for preventing, treating or ameliorating sarcoma in a subject in need thereof comprises, administering an amount of Compound 1 effective to inhibit or reduce tubulin polymerization in the subject is described herein.

In one aspect, a method for preventing, treating or ameliorating sarcoma in a subject in need thereof comprises, administering an amount of Compound 1 effective to inhibit or reduce BMI-1 function in the subject is described herein.

In a specific aspect, a method for preventing, treating or ameliorating sarcoma in a subject in need thereof as described herein inhibits or reduces tubulin polymerization by about 5%, 10%, 15%, 20%, 25%, 30%, 35%, 40%, 45%, 50%, 55%, 60%, 65%, 80%, 85%, 90%, 95%, or 100% relative to tubulin polymerization prior to administration of Compound 1 to the subject, as assessed by methods well known in the art.

In a specific aspect, a method for preventing, treating or ameliorating sarcoma in a subject in need thereof as described herein inhibits or reduces BMI-1 function by about 5%, 10%, 15%, 20%, 25%, 30%, 35%, 40%, 45%, 50%, 55%, 60%, 65%, 80%, 85%, 90%, 95%, or 100% relative to BMI-1 function prior to administration of Compound 1 to the subject, as assessed by methods well known in the art.

In a specific aspect, a method for preventing, treating or ameliorating sarcoma in a subject in need thereof as described herein inhibits or reduces tubulin polymerization in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to tubulin polymerization prior to administration of Compound 1 to the subject, as assessed by methods well known in the art.

In a specific aspect, a method for preventing, treating or ameliorating sarcoma in a subject in need thereof as described herein inhibits or reduces BMI-1 function in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to BMI-1 function prior to administration of Compound 1 to the subject, as assessed by methods well known in the art.

In various aspects, a method for preventing, treating or ameliorating sarcoma in a subject in need thereof as described herein reduces the concentration of BMI-1 in a subject as assessed by methods well known in the art, e.g., ELISA.

In one aspect, a method for preventing, treating or ameliorating sarcoma in a subject in need thereof comprises, administering an amount of Compound 1 effective to inhibit proliferation or reduce an in vitro or in vivo proliferating cell or cell line population in the subject is described herein.

In a specific aspect, a method for preventing, treating or ameliorating sarcoma in a subject in need thereof as described herein inhibits proliferation or reduces an in vitro or in vivo proliferating cell or cell line population in the subject by about 5%, 10%, 15%, 20%, 25%, 30%, 35%, 40%, 45%, 50%, 55%, 60%, 65%, 80%, 85%, 90%, 95%, or 100% relative to proliferation or in vitro or in vivo proliferating cell or cell line population in the subject prior to administration of Compound 1 to the subject, as assessed by methods well known in the art.

In a specific aspect, a method for preventing, treating or ameliorating sarcoma in a subject in need thereof as described herein inhibits proliferation or reduces an in vitro or in vivo proliferating cell or cell line population in the subject in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to proliferation or in vitro or in vivo proliferating cell or cell line population in the subject prior to administration of Compound 1 to the subject, as assessed by methods well known in the art.

In various aspects, a method for preventing, treating or ameliorating sarcoma in a subject in need thereof as described herein inhibits proliferation or reduces an in vitro or in vivo proliferating cell or cell line population in a subject as assessed by methods well known in the art, e.g., ELISA.

In one aspect, a method for preventing, treating or ameliorating sarcoma in a subject in need thereof comprises, administering an amount of Compound 1 effective to inhibit proliferation or reduce an in vitro or in vivo proliferating cell or cell line population in the subject in combination with another therapy (e.g., one or more additional therapies that do not comprise Compound 1, or that comprise a different anti-proliferative agent) to a subject in need thereof is described herein.

Such methods may involve administering Compound 1 prior to, concurrent with, or subsequent to administration of the additional therapy. In certain aspects, such methods have an additive or synergistic effect.

In a specific aspect, presented herein is a method for preventing, treating or ameliorating sarcoma in a subject in need thereof comprising, administering to a subject in need thereof an effective amount of Compound 1 and an effective amount of another therapy.

One aspect described herein includes a hematologic cancer that can be prevented, treated or ameliorated in accordance with the methods provided herein include, but are not limited to, sarcoma.

In one aspect, presented herein is a method for preventing, treating or ameliorating sarcoma, comprising: (a) administering to a subject in need thereof one or more doses of Compound 1 or a pharmaceutically acceptable salt or pharmaceutical composition thereof a pharmaceutical composition thereof; and (b) monitoring the concentration of certain biomarkers, before and/or after step (a).

In a specific aspect, the monitoring step (b) is carried out before and/or after a certain number of doses (e.g., 1, 2, 4, 6, 8, 10, 12, 14, 15, or 29 doses, or more doses; 2 to 4, 2 to 8, 2 to 20 or 2 to 30 doses) or a certain time period (e.g., 1, 2, 3, 4, 5, 6, or 7 days; or 1, 2, 3, 4, 5, 10, 15, 20, 30, 40, 45, 48, or 50 weeks) of administering Compound 1 or a pharmaceutically acceptable salt or pharmaceutical composition thereof.

In a specific aspect, one or more of these monitoring parameters are detected prior to administration of Compound 1 or a pharmaceutically acceptable salt or pharmaceutical composition thereof to the subject.

In a specific aspect, a decrease in the proliferation of an in vitro or in vivo proliferating cell or cell line population following administration of Compound 1 or a pharmaceutically acceptable salt or pharmaceutical composition thereof indicates that the course of treatment is effective for preventing, treating or ameliorating the sarcoma.

In a specific aspect, a change in the proliferation of an in vitro or in vivo proliferating cell or cell line population following administration of Compound 1 or a pharmaceutically acceptable salt or pharmaceutical composition thereof may indicate that the dosage, frequency and/or length of administration of Compound 1 or a pharmaceutically acceptable salt or pharmaceutical composition thereof may be adjusted (e.g., increased, reduced or maintained).

In a specific aspect, the concentration of certain biomarkers in biological specimens of a subject is monitored before, during and/or after a course of treatment for sarcoma involving the administration of Compound 1 or a pharmaceutically acceptable salt or pharmaceutical composition thereof to the subject.

The dosage, frequency and/or length of administration of Compound 1 or a pharmaceutically acceptable salt or pharmaceutical composition thereof to a subject might be modified as a result of the proliferation of an in vitro or in vivo proliferating cell or cell line population.

Alternatively, the changes in these monitoring parameters (e.g., concentration of certain biomarkers) might indicate that the course of treatment involving the administration of the Compound 1 or a pharmaceutically acceptable salt or pharmaceutical composition thereof is effective in preventing, treating or ameliorating the sarcoma.

The concentration of certain biomarkers in a subject may be detected by any technique known to one of skill in the art. In certain aspects, the method for detecting the concentration of certain biomarkers of a subject comprises obtaining a biological sample (e.g., tissue or fluid sample) from the subject and detecting the concentration of the biomarkers in the biological sample (e.g., from plasma, serum, urine, or any other biofluids), that has been subjected to certain types of treatment (e.g., centrifugation), and detection by use of immunological techniques, such as ELISA.

In a specific aspect, an ELISA assay, as described herein, may be used to detect the concentration of the biomarkers in a biological sample (e.g., from plasma, serum, urine, or any other biofluids) that has been subjected to certain types of treatment (e.g., centrifugation). Other techniques known in the art that may be used to detect the concentration of the biomarkers in a biological sample include multiplex or proteomic assays.

In specific aspects, the methods for preventing, treating or ameliorating sarcoma provided herein alleviate or manage one, two or more symptoms associated with the sarcoma. Alleviating or managing one, two or more symptoms of the sarcoma may be used as a clinical endpoint for efficacy of Compound 1 or a pharmaceutically acceptable salt or pharmaceutical composition thereof for preventing, treating or ameliorating the sarcoma. In some aspects, the methods for preventing, treating or ameliorating the sarcoma provided herein reduce the duration and/or severity of one or more symptoms associated with the sarcoma. In some aspects, the methods for preventing, treating or ameliorating the sarcoma provided herein inhibit the onset, progression and/or recurrence of one or more symptoms associated with the sarcoma. In some aspects, the methods for treating the sarcoma provided herein reduce the number of symptoms associated with the sarcoma.

In certain aspects, the methods for preventing, treating or ameliorating sarcoma provided herein prolong or delay the GUS or late GUS phase of the cell cycle (i.e., the period between the late checkpoint (resting or pre-DNA synthesis phase), and the early DNA synthesis phase). In other aspects, the methods for preventing, treating or ameliorating sarcoma provided herein prolong or delay the S or G2/M phase of the cell cycle (i.e., the period between DNA synthesis and the early division phase).

In some aspects, the methods for preventing, treating or ameliorating sarcoma provided herein reduce, ameliorate, or alleviate the severity of the sarcoma and/or one or more symptoms thereof.

In other aspects, the methods for preventing, treating or ameliorating sarcoma provided herein reduce hospitalization (e.g., the frequency or duration of hospitalization) of a subject diagnosed with the sarcoma.

In certain aspects, the methods provided herein increase the survival of a subject diagnosed with sarcoma. In specific aspects, the methods provided herein increase the survival of a subject diagnosed with sarcoma by about 6 months or more, about 7 months or more, about 8 months or more, about 9 months or more, or about 12 months or more.

In particular aspects, the methods for preventing, treating or ameliorating sarcoma provided herein inhibit or reduce the progression of the sarcoma, or one or more symptoms associated therewith. In specific aspects, the methods for preventing, treating or ameliorating sarcoma provided herein enhance or improve the therapeutic effect of another therapy (e.g., an anti-cancer agent, radiation, drug therapy, such as chemotherapy, anti-androgen therapy, or surgery). In certain aspects, the methods for preventing, treating or ameliorating sarcoma provided herein involve the use of Compound 1 or a pharmaceutically acceptable salt or pharmaceutical composition thereof as an adjuvant therapy.

In particular aspects, the methods for preventing, treating or ameliorating sarcoma provided herein reduce the mortality of subjects diagnosed with the sarcoma. In certain aspects, the methods for preventing, treating or ameliorating sarcoma provided herein increase the number of subjects in remission or decrease the hospitalization rate. In other aspects, the methods for preventing, treating or ameliorating sarcoma provided herein prevent the development, onset or progression of one or more symptoms associated with the sarcoma.

In particular aspects, the methods for preventing, treating or ameliorating sarcoma provided herein increase symptom-free survival of sarcoma subjects. In some aspects, the methods for preventing, treating or ameliorating sarcoma provided herein do not cure the sarcoma in subjects, but prevent the progression or worsening of the disease. In some aspects, the methods for preventing, treating or ameliorating sarcoma provided herein improve the subject's quality of life.

In certain aspects, the methods for preventing, treating or ameliorating sarcoma provided herein increase the cancer-free survival rate of subjects diagnosed with the cancer. In some aspects, the methods for preventing, treating or ameliorating sarcoma provided herein increase relapse-free survival. In certain aspects, the methods for preventing, treating or ameliorating sarcoma provided herein increase the number of subjects in remission. In other aspects, the methods for preventing, treating or ameliorating sarcoma provided herein increase the length of remission in subjects.

Treatment Population

In one aspect, a subject treated for sarcoma in accordance with the methods provided herein is a human who has or is diagnosed with sarcoma. In another aspect, a subject treated for sarcoma in accordance with the methods provided herein is a human predisposed or susceptible to sarcoma. In another aspect, a subject treated for sarcoma in accordance with the methods provided herein is a human at risk of developing sarcoma. In another aspect, a subject treated for sarcoma in accordance with the methods provided herein is a human having a genetic or somatic mutation placing the subject at risk or predisposition for developing sarcoma.

In one aspect, a subject treated for sarcoma in accordance with the methods provided herein is a human infant. In another aspect, a subject treated for sarcoma in accordance with the methods provided herein is a human toddler. In another aspect, a subject treated for sarcoma in accordance with the methods provided herein is a human child. In another aspect, a subject treated for sarcoma in accordance with the methods provided herein is a human adult. In another aspect, a subject treated for sarcoma in accordance with the methods provided herein is a middle-aged human. In another aspect, a subject treated for sarcoma in accordance with the methods provided herein is an elderly human.

In certain aspects, a subject treated for cancer in accordance with the methods provided herein has sarcoma metastasized to other areas of the body, such as the bones, lung and liver. In certain aspects, a subject treated for sarcoma in accordance with the methods provided herein is in remission from the sarcoma. In some aspects, the subject treated for sarcoma in accordance with the methods provided herein had a recurrence of the sarcoma. In certain aspects, a subject treated in accordance with the methods provided herein is experiencing recurrence of one or more symptoms associated with the sarcoma.

In certain aspects, a subject treated for sarcoma in accordance with the methods provided herein is i). a human toddler that is in an age range of from about 1 to about 5 years old; ii). a human child that is in an age range of from about 5 to 10 years old; or, from about 10 to about 18 years old; ii). a human adult that is in an age range of from about 18 to about 30 years old; or, from about 25 to about 35 years old; or, from about 35 to about 45 years old ii). a middle-aged human adult that is in an age range of from about 40 to about 55 years old; or, from about 50 to about 65 years old ii). a human adult that is in an age range of from about 60 to about 75 years old, ii). a human toddler that is about 70 to about 85 years old, about 80 to about 90 years old, about 90 to about 95 years old or about 95 to about 100 years old, or any age in between.

In a specific aspect, a subject treated for sarcoma in accordance with the methods provided herein is a human that is 18 years old or older. In a particular aspect, a subject treated for sarcoma in accordance with the methods provided herein is a human child that is between the age of 1 year old to 18 years old. In a certain aspect, a subject treated for sarcoma in accordance with the methods provided herein is a human that is between the age of 12 years old and 18 years old. In a certain aspect, the subject is a male human. In another aspect, the subject is a female human. In one aspect, the subject is a female human that is not pregnant or is not breastfeeding. In one aspect, the subject is a female that is pregnant or will/might become pregnant, or is breast feeding.

As used herein, the term “human infant” refers to a newborn to 1 year old human.

As used herein, the term “human toddler” refers to a human that is 1 year to 5 years old.

As used herein, the term “human child” refers to a human that is 5 years to 18 years old.

As used herein, the term “human adult” refers to a human that is 18 years or older.

As used herein, the term “middle-aged human” refers to a human between the ages of 40 and 65.

As used herein, the term “elderly human” refers to a human 65 years or older.

In particular aspects, a subject treated for sarcoma in accordance with the methods provided herein is a human that is in an immunocompromised state or immunosuppressed state. In certain aspects, a subject treated for sarcoma in accordance with the methods provided herein is a human receiving or recovering from immunosuppressive therapy. In certain aspects, a subject treated for sarcoma in accordance with the methods provided herein is a human that has or is at risk of getting sarcoma. In certain aspects, a subject treated for sarcoma in accordance with the methods provided herein is a human who is, will or has undergone surgery, drug therapy, such as chemotherapy, hormonal therapy and/or radiation therapy.

In some aspects, a subject treated for sarcoma in accordance with the methods provided herein is administered Compound 1 or a pharmaceutically acceptable salt or pharmaceutical composition thereof, or a combination therapy before any adverse effects or intolerance to therapies other than Compound 1 develops. In some aspects, a subject treated for sarcoma in accordance with the methods provided herein is a refractory subject. In certain aspects, a refractory subject is a subject refractory to a standard therapy (e.g., surgery, radiation and/or drug therapy such as chemotherapy). In certain aspects, a subject with sarcoma is refractory to a therapy when the sarcoma has not significantly been eradicated and/or the one or more symptoms have not been significantly alleviated. The determination of whether a subject refractory can be made either in vivo or in vitro by any method known in the art for assaying the effectiveness of a treatment of sarcoma, using art-accepted meanings of “refractory” in such a context.

In some aspects, a subject treated for sarcoma in accordance with the methods provided herein is a human that has proven refractory to therapies other than treatment with Compound 1 or a pharmaceutically acceptable salt or pharmaceutical composition thereof, but is no longer on these therapies. In certain aspects, a subject treated for sarcoma in accordance with the methods provided herein is a human already receiving one or more conventional anti-cancer therapies, such as surgery, drug therapy such as chemotherapy, anti-androgen therapy or radiation. Among these subjects are refractory subjects, subjects who are too young for conventional therapies, and subjects with recurring sarcoma despite treatment with existing therapies.

In some aspects, a subject treated for sarcoma in accordance with the methods provided herein is a human susceptible to adverse reactions to conventional therapies. In some aspects, a subject treated for sarcoma in accordance with the methods provided herein is a human that has not received a therapy, e.g., drug therapy such as chemotherapy, surgery, anti-androgen therapy or radiation therapy, prior to the administration of Compound 1 or a pharmaceutically acceptable salt or pharmaceutical composition thereof. In other aspects, a subject treated for sarcoma in accordance with the methods provided herein is a human that has received a therapy prior to administration of Compound 1 or a pharmaceutically acceptable salt or pharmaceutical composition thereof. In some aspects, a subject treated for sarcoma in accordance with the methods provided herein is a human that has experienced adverse side effects to the prior therapy or the prior therapy was discontinued due to unacceptable levels of toxicity to the human.

Dosage and Administration

In accordance with the methods for preventing, treating or ameliorating sarcoma provided herein, Compound 1 or a pharmaceutically acceptable salt or pharmaceutical composition thereof can be administered to a subject in need thereof by a variety of routes in amounts which result in a beneficial or therapeutic effect. Compound 1 or a pharmaceutically acceptable salt or pharmaceutical composition thereof may be orally administered to a subject in need thereof in accordance with the methods for preventing, treating or ameliorating sarcoma provided herein. The oral administration of Compound 1 or a pharmaceutically acceptable salt or pharmaceutical composition thereof may facilitate subjects in need of such treatment complying with a regimen for taking Compound 1 or a pharmaceutically acceptable salt or pharmaceutical composition thereof. Thus, in a specific aspect, Compound 1 or a pharmaceutically acceptable salt or pharmaceutical composition thereof is administered orally to a subject in need thereof. In another aspect, Compound 1 or a pharmaceutically acceptable salt or pharmaceutical composition thereof provided herein can be administered orally, with or without food or water.

Other routes of administration include, but are not limited to, intravenous, intradermal, intrathecal, intramuscular, subcutaneous, intranasal, inhalation, transdermal, topical, transmucosal, intracranial, epidural and intra-synovial. In one aspect, Compound 1 or a pharmaceutically acceptable salt or pharmaceutical composition thereof is administered systemically (e.g., parenterally) to a subject in need thereof. In one aspect, Compound 1 or a pharmaceutically acceptable salt or pharmaceutical composition thereof is administered via a route that permits Compound 1 or a pharmaceutically acceptable salt or pharmaceutical composition thereof to cross the blood-brain barrier (e.g., orally).

In accordance with the methods for preventing, treating or ameliorating sarcoma provided herein that involve administration of Compound 1 or a pharmaceutically acceptable salt or pharmaceutical composition thereof in combination with one or more additional therapies, Compound 1 or a pharmaceutically acceptable salt or pharmaceutical composition thereof and one or more additional therapies may be administered by the same route or a different route of administration.

The dosage and frequency of administration of Compound 1 or a pharmaceutically acceptable salt or pharmaceutical composition thereof is administered to a subject in need thereof in accordance with the methods for preventing, treating or ameliorating sarcoma provided herein will be efficacious while minimizing any side effects. The exact dosage and frequency of administration of Compound 1 or a pharmaceutically acceptable salt or pharmaceutical composition thereof can be determined by a practitioner, in light of factors related to the subject that requires treatment.

Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of Compound 1 or a pharmaceutically acceptable salt or pharmaceutical composition thereof may be adjusted over time to provide an effective amount of Compound 1 or a pharmaceutically acceptable salt or pharmaceutical composition thereof or to maintain the desired effect.

As described herein, the methods for preventing, treating or ameliorating sarcoma in a subject in need thereof presented herein comprises, administering to the subject an effective amount of Compound 1 or a pharmaceutically acceptable salt or pharmaceutical composition thereof.

In another aspect, the term “effective amount” refers to that amount of Compound 1 administered as a monotherapy to a patient, which effective amount is in a range of from about 0.001 mg/Kg/day to about 500 mg/Kg/day, or about 0.01 mg/Kg/day to about 500 mg/Kg/day, or about 0.1 mg to about 500 mg/Kg/day, or about 1.0 mg/day to about 500 mg/Kg/day, in single, divided, or a continuous dose for a patient or subject having a weight in a range of between about 40 to about 200 Kg (which dose may be adjusted for patients or subjects above or below this range, particularly children under 40 Kg). Dosing may be administered as a dose per kilogram, a dose per meter squared or a flat dose expressed in a unit of weight (e.g., milligrams, grams).

In another aspect, the effective amount is a dose administered to the subject that may be increased or decreased depending on subject response. The effective amount for the subject will also depend upon various factors, including the body weight, size and health of the subject. The typical adult subject is expected to have a median weight in a range of between about 60 to about 100 Kg. Accordingly, an effective amount for a given patient may be determined according to the skill and judgment of the clinician.

In one aspect, daily monotherapy doses may be adjusted based upon the weight of the subject or patient, wherein Compound 1 may be formulated for delivery as a monotherapy at about 0.02, 0.025, 0.03, 0.05, 0.06, 0.075, 0.08, 0.09, 0.10, 0.15, 0.20, 0.25, 0.30, 0.35, 0.40, 0.45, 0.50, 0.55, 0.60, 0.65, 0.75, 0.80, 0.90, 1.0, 1.10, 1.20, 1.25, 1.50, 1.75, 2.0, 2.5, 3.0, 3.5, 4.0, 4.5, 5.0, 5.5, 10, 20, 50, 75 or 100 mg/Kg/day or any range in between.

In another aspect, a daily dose may be adjusted based upon the weight of the subject or patient and administered as a single, divided, or continuous dose.

In another aspect, a daily dose of Compound 1 may be administered more than once per day, as in once, twice, three times, or more per day.

In another aspect, a dose of Compound 1 may be administered more than once per week, as in once, twice, three times, or more per week.

In another aspect, the effective amount may be a dose administered to the subject twice per week on different days, wherein the second dose in a week follows the first by three days, and wherein the first dose in a following week follows the second dose in a preceding week by four days. In another embodiment, a subject may be administered one or more doses of an effective amount of Compound 1, wherein the effective amount may not be the same for each dose.

In one aspect, an effective amount of Compound 1 may range from about 0.001 mg/Kg/day to about 500 mg/Kg/day. Within the scope described herein, the “effective amount” of Compound 1 for use in the manufacture of a medicament or in a method for treating sarcoma in a subject in need thereof, is intended to include an amount in a range of from about 0.1 ng to about 3500 mg administered daily; from about 0.1 μg to about 3500 mg administered daily; from about 0.1 mg to about 3500 mg administered daily; from about 1 mg to about 3500 mg administered daily; from about 1 mg to about 3000 mg administered daily; from about 0.05 mg to about 1500 mg administered daily; from about 0.5 mg to about 1500 mg administered daily; from about 1 mg to about 1500 mg administered daily; from about 5 mg to about 1500 mg administered daily; from about 10 mg to about 600 mg administered daily; from about 0.5 mg to about 2000 mg administered daily; or, an amount in a range of from about 5.0 mg to about 1500 mg administered daily.

In another aspect, the effective amount of Compound 1 is in a range of from about 0.1 ng to about 3500 mg.

In one aspect, the effective amount of Compound 1 can be estimated initially by results from cell culture assays or from human or relevant animal models, such as the mouse, chimpanzee, marmoset or tamarin animal model. Relevant animal models may also be used to determine the appropriate concentration range and route of administration. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between the toxic and therapeutic effect is referred to as the therapeutic index, and can be expressed as the ratio, LD₅₀/ED₅₀. In another aspect, the effective amount is such that a large therapeutic index is achieved. In another aspect, the dose administered results in a range of plasma concentrations that include an ED₅₀ with little or no toxicity. The dosage may vary within this range depending upon the dosage form employed, sensitivity of the patient, and the route of administration.

More specifically, the concentration-biological effect (pharmacodynamic) relationship observed with regard to Compound 1 suggests a target plasma concentration ranging from about 0.001 μg/mL to about 50 μg/mL, from about 0.01 μg/mL to about 20 μg/mL, from about 0.05 μg/mL to about 10 μg/mL, or from about 0.1 μg/mL to about 5 μg/mL. To achieve such plasma concentrations, Compound 1 may be administered at doses that vary from 0.001 μg to 100,000 mg, depending upon the route of administration in single, divided, or continuous doses for a patient weighing between about 40 to about 100 kg (which dose may be adjusted for patients above or below this weight range, particularly for children under 40 kg).

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose selected from a dose in a range of from about 50 mg to about 400 mg, from about 100 mg to about 200 mg, from about 125 mg to about 175 mg, from about 100 mg to about 300 mg, from about 100 mg to about 400 mg, from about 150 mg to about 200 mg, from about 150 mg to about 300 mg, from about 150 mg to about 400 mg, from about 200 mg to about 300 mg, from about 225 mg to about 275 mg, from about 225 mg to about 300 mg, from about 275 mg to about 300 mg, from about 200 mg to about 225 mg, from about 200 mg to about 275 mg, from about 200 mg to about 400 mg, from about 250 mg to about 300 mg, from about 250 mg to about 400 mg, from about 250 mg to about 350 mg, and the like, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 50 mg to about 400 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 100 mg to about 200 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 125 mg to about 175 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 125 mg to about 200 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 175 mg to about 200 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 100 mg to about 125 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 100 mg to about 175 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 100 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 100 mg to about 400 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 150 mg to about 200 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 150 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 150 mg to about 400 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 200 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 225 mg to about 275 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 225 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 275 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 200 mg to about 225 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 200 mg to about 275 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 200 mg to about 400 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 250 mg to about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 250 mg to about 400 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose in a range of from about 250 mg to about 350 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose selected from the group consisting of about 50 mg, about 100 mg, about 125 mg, about 150 mg, about 175 mg, about 200 mg, about 250 mg, about 300 mg, about 350 mg, about 400 mg, and about 450 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose selected from the group consisting of about 100 mg, about 125 mg, about 150 mg, about 175 mg, about 200 mg, about 225 mg, about 250 mg, about 275 mg, and about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose selected from the group consisting of about 125 mg, about 150 mg, about 175 mg, about 225 mg, about 250 mg, about 275 mg, and about 300 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 100 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 125 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 150 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 175 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 200 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 225 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 250 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 275 mg, administered orally twice per week.

In another aspect, a method for preventing, treating or ameliorating sarcoma in a subject in need thereof comprises the administration of an effective amount of Compound 1 or a pharmaceutically acceptable salt or pharmaceutical composition thereof to the subject, wherein the effective amount is a dose of about 300 mg, administered orally twice per week.

In another aspect, a method for preventing, treating or ameliorating sarcoma in a subject in need thereof comprises the administration of an effective amount of Compound 1 or a pharmaceutical composition thereof to the subject, wherein the effective amount is a dosage that is expressed as mg per meter squared (mg/m²). The mg/m² for Compound 1 may be determined, for example, by multiplying a conversion factor for an animal (e.g., mouse, rat, hamster, guinea pig, dog, monkey and man) by an animal dose in mg per kilogram (mg/kg) to obtain the dose in mg/m² for the human dose equivalent, where the following allometric conversion factors (assuming allometric conversions are equal for all drugs) may be used, for example: Mouse=3, Hamster=4.1, Rat=6, Guinea Pig=7.7. (based on Freireich et al., Cancer Chemother. Rep. 50(4):219-244 (1966)).

The height and weight of a human may be used to calculate a human body surface area applying Boyd's Formula of Body Surface Area.

In another aspect, an average weight for a human is generally considered to be 70 kg. However, ideal body weight-based dosing may be superior to currently recommended total body weight-based regimen in adult subjects. The estimated ideal body weight (IBW)(kg) for males is calculated as: 50 kg+2.3 kg for each inch over 5 feet. For females, the estimated ideal body weight is calculated as: 45.5 kg+2.3 kg for each inch over 5 feet.

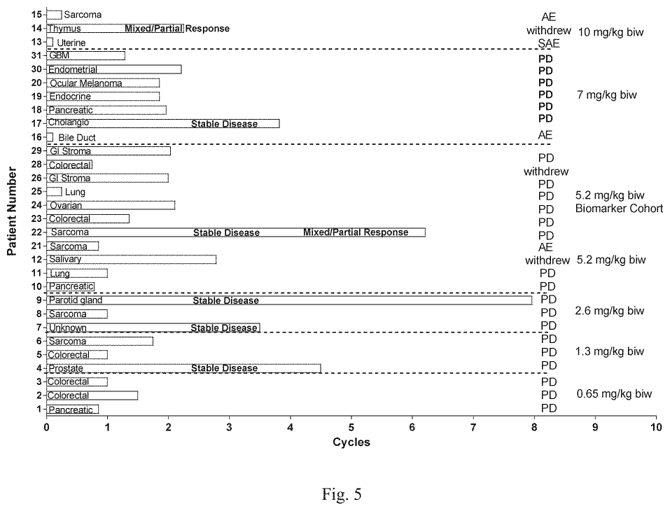

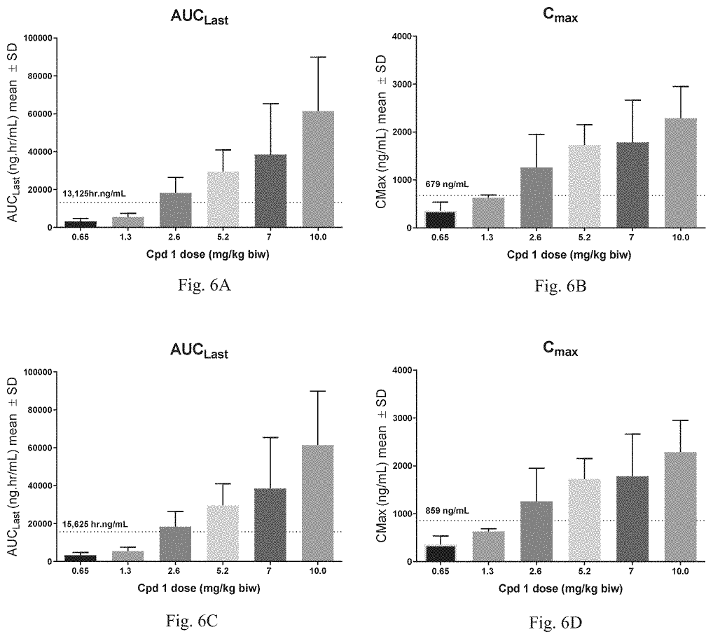

In contrast to use of allometric conversion factors, FIGS. 6A-D summarize the pharmacokinetics of Compound 1 administration in humans, showing that a conversion factor for Compound 1 based on free drug in humans compared to free drug in mice can be derived to estimate the effective target AUC and Cₘₐₓ, thus accounting for the differences in free drug between these species.

As shown in Table A1 below, FIG. 6A shows the measured human exposure for AUC at different doses. The dashed line indicates the minimum estimated AUC target of at least 13,125 hr-ng/mL based on the exposure in the mouse at 10 mg/kg, taking into account free drug. As shown by the dashed line, this predicts that the estimated dose in a human patient of about 2.0 mg/kg (biw) or higher of Compound 1 would be effective.

As shown in Table A1 below, FIG. 6B shows the measured human exposure for Cₘₐₓ at different doses. The dashed line indicates that the minimum estimated Cₘₐₓ target of at least 688 ng/mL based on the exposure in the mouse at 10 mg/kg, taking into account free drug. As shown by the dashed line, this predicts that the estimated dose in a human patient of about 1.4 mg/kg (biw) or higher of Compound 1 would be effective.

As shown in Table A1 below, FIG. 6C shows the measured human exposure for AUC at different doses. The dashed line indicates that the minimum estimated AUC target of at least 15,625 hr-ng/mL based on the exposure in the mouse at 12.5 mg/kg, taking into account free drug. As shown by the dashed line, this predicts that the estimated dose in a human patient of about 2.3 mg/kg (biw) or higher of Compound 1 would be effective.

As shown in Table A1 below, FIG. 6D shows the measured human exposure for Cₘₐₓ at different doses. The dashed line indicates that the minimum estimated Cₘₐₓ target of at least 859 ng/mL based on the exposure in the mouse at 12.5 mg/kg, taking into account free drug. As shown by the dashed line, this predicts that the estimated dose in a human patient of about 1.8 mg/kg (biw) or higher of Compound 1 would be effective.

Free drug concentration differs in humans compared to mice by a ratio of 8:5.

TABLE A1

| | | | Predicted dose in | |
|---|---|---|---|---|
| | | | human | |
| Dose in | Free Drug in mouse | | mg/kg | mg/kg |
| Mouse | AUC | Cmax | based | based on |
| (mg/kg) | (hr-ng/mL) | (ng/mL) | on AUC | Cmax |
| 10 | 13125 | 688 | 2.0 | 1.4 |
| 12.5 | 15625 | 859 | 2.3 | 1.8 |

As shown in Table A2 below, if free drug concentration were not taken into account, the estimated target AUC and Cₘₐₓ as total drug in humans and mice would be the same.

As shown in Table A2 below, the preclinical dose of 10 mg/kg administered orally twice per week to mice, as used herein, would result in a predicted estimated minimum effective human dose range between about 3.2 mg/kg to about 2.3 mg/kg biw to maintain AUC and Cₘₐₓ, respectively, above the target concentrations for an expected therapeutic effect.

As shown in Table A2 below, the preclinical dose of 12.5 mg/kg administered orally twice per week to mice, as used herein, would result in a predicted estimated minimum effective human dose range between about 4.2 mg/kg to about 3.2 mg/kg biw to maintain AUC and Cₘₐₓ, respectively, above the target concentrations for an expected therapeutic effect.

TABLE A2

| | | | Predicted Dose in | |
|---|---|---|---|---|
| | Total Drug in | | Human | |
| Dose in | Mouse | | mg/kg | mg/kg |
| Mouse | AUC | Cmax | based | based on |
| (mg/kg) | (hr-ng/mL) | (ng/mL) | on AUC | Cmax |
| 10 | 21000 | 1100 | 3.2 | 2.3 |
| 12.5 | 26250 | 1375 | 4.2 | 3.2 |

For a 70 kg human subject, based on targeting free drug at 12.5 mg/kg in the mouse, the corresponding minimum effective amount of Compound 1 for use as a monotherapy in humans is a dose in a range of about 2.3 mg/kg to about 1.8 (for AUC and Cₘₐₓ, respectively), resulting in an amount of from about 162 mg to about 124 mg, administered orally twice per week.

For a 70 kg human subject, based on targeting total drug at 12.5 mg/kg in the mouse, the corresponding minimum effective amount of Compound 1 for use as a monotherapy in humans is a dose in a range of about 4.2 to about 3.2 mg/kg (for AUC and Cₘₐₓ, respectively), resulting in an amount of from about 290 mg to about 226 mg, administered orally twice per week.

In specific aspects, a method for preventing, treating or ameliorating sarcoma in a subject in need thereof comprises the administration of an effective amount of Compound 1 or a pharmaceutical composition thereof to the subject, wherein the effective amount is an amount in the range of from about 0.1 mg/m² to about 1000 mg/m², or any range in between.

In one aspect, a method for preventing, treating or ameliorating sarcoma in a subject in need thereof comprises the administration of an effective amount of Compound 1 or a pharmaceutical composition thereof to the subject, wherein the effective amount is a dosage that achieves a target mean plasma concentration of Compound 1 in a subject with sarcoma or an animal model with a pre-established sarcoma.

In another aspect, a method for preventing, treating or ameliorating sarcoma in a subject in need thereof comprises the administration of an effective amount of Compound 1 or a pharmaceutical composition thereof to the subject, wherein the effective amount is a dosage that achieves a mean maximum plasma concentration (Cₘₐₓ) of Compound 1 in a 24 hour period in a range of from approximately 0.1 hr-μg/mL to approximately 1.0 hr-μg/mL, approximately 0.2 hr-μg/mL to approximately 1.0 hr-μg/mL, approximately 0.3 hr-μg/mL to approximately 1.0 hr-μg/mL, approximately 0.4 hr-μg/mL to approximately 1.0 hr-μg/mL, approximately 0.5 hr-μg/mL to approximately 1.0 hr-μg/mL, approximately 0.6 hr-μg/mL to approximately 1.0 hr-μg/mL, approximately 0.7 hr-μg/mL to approximately 1.0 hr-μg/mL, approximately 0.8 hr-μg/mL to approximately 1.0 hr-μg/mL, approximately 0.9 hr-μg/mL to approximately 1.0 hr-μg/mL, approximately 3 hr-μg/mL to approximately 70 hr-μg/mL, from approximately 3 hr-μg/mL to approximately 60 hr-μg/mL, from approximately 3 hr-μg/mL to approximately 50 hr-μg/mL, from approximately 3 hr-μg/mL to approximately 40 hr-μg/mL, from approximately 3 hr-μg/mL to approximately 30 hr-μg/mL, from approximately 3 hr-μg/mL to approximately 20 hr-μg/mL, from approximately 3 hr-μg/mL to approximately 10 hr-μg/mL, and the like, or any range in between, in a subject with the sarcoma or an animal model with a pre-established sarcoma.

In another aspect, a method for preventing, treating or ameliorating sarcoma in a subject in need thereof comprises the administration of an effective amount of Compound 1 or a pharmaceutical composition thereof to the subject, wherein the effective amount is a dosage that achieves a mean plasma concentration of Compound 1 in a 24 hour period of approximately 0.1 hr-μg/mL, approximately 0.2 hr-μg/mL, approximately 0.3 hr-μg/mL, approximately 0.4 hr-μg/mL, approximately 0.5 hr-μg/mL, approximately 0.6 hr-μg/mL, approximately 0.7 hr-μg/mL, approximately 0.8 hr-μg/mL, approximately 0.9 hr-μg/mL, approximately 1.0 hr-μg/mL, approximately 1.1 hr-μg/mL, approximately 1.2 hr-μg/mL, approximately 1.3 hr-μg/mL, approximately 1.4 hr-μg/mL, approximately 1.5 hr-μg/mL, approximately 1.6 hr-μg/mL, approximately 1.7 hr-μg/mL, approximately 1.8 hr-μg/mL, approximately 1.9 hr-μg/mL, approximately 2.0 hr-μg/mL, approximately 2.1 hr-μg/mL, approximately 2.2 hr-μg/mL, approximately 2.3 hr-μg/mL, approximately 2.4 hr-μg/mL, approximately 2.5 hr-μg/mL, approximately 2.6 hr-μg/mL, approximately 2.7 hr-μg/mL, approximately 2.8 hr-μg/mL, approximately 2.9 hr-μg/mL, approximately 3.0 hr-μg/mL, approximately 10 hr-μg/mL, approximately 20 hr-μg/mL, approximately 30 hr-μg/mL, approximately 40 hr-μg/mL, approximately 50 hr-μg/mL, approximately 60 hr-μg/mL, approximately 70 hr-μg/mL, and the like, or any range in between, in a subject with the sarcoma or an animal model with a pre-established sarcoma.

To achieve such plasma concentrations, a dose described herein of Compound 1 or a pharmaceutical composition thereof may be administered. In certain aspects, subsequent doses of Compound 1 or a pharmaceutical composition thereof may be adjusted accordingly based on the mean plasma concentrations of Compound 1 achieved with a dose of Compound 1 or a pharmaceutical composition thereof administered to the subject.

In specific aspects, a method for preventing, treating or ameliorating sarcoma in a subject in need thereof comprises the administration of an effective amount of Compound 1 or a pharmaceutical composition thereof to the subject, wherein the effective amount is a dosage that achieves a reduced target mean plasma concentration of one or more biomarkers in a subject with the sarcoma or an animal model with a pre-established sarcoma.

In particular aspects, a method for preventing, treating or ameliorating sarcoma in a subject in need thereof comprises the administration of an effective amount of Compound 1 or a pharmaceutical composition thereof to the subject, wherein the effective amount is a dosage that achieves the desired tissue to mean plasma concentration ratios of Compound 1 or a pharmaceutical composition thereof as determined, e.g., by any imaging techniques known in the art, in a subject with the sarcoma or an animal model with a pre-established sarcoma.

In some aspects, a method for preventing, treating or ameliorating sarcoma in a subject in need thereof comprises the administration of an effective amount of Compound 1 or a pharmaceutical composition thereof to the subject, wherein the effective amount may or may not be the same for each dose. In particular aspects, a first (i.e., initial) dose of Compound 1 or a pharmaceutical composition thereof is administered to a subject in need thereof for a first period of time, followed by a second (i.e., loading) dose of Compound 1 or a pharmaceutical composition thereof is administered to the subject for a second period of time and, subsequently, a third (i.e., maintenance) dose of Compound 1 or a pharmaceutical composition thereof is administered to the subject for a second period of time. The first dose may be more than the second dose, or the first dose may be less than the second dose. In similar fashion, the third dose of Compound 1 or a pharmaceutical composition thereof may be more or less than the second dose and more or less than the first dose.

In some aspects, the dosage amounts described herein refer to total amounts administered; that is, if more than one Compound is administered, then, in some aspects, the dosages correspond to the total amount administered. In a specific aspect, oral compositions contain about 5% to about 95% of Compound 1 by weight.

The length of time that a subject in need thereof is administered Compound 1 or a pharmaceutical composition thereof in accordance with a method for preventing, treating or ameliorating sarcoma in a subject in need thereof will be the time period that is determined by cancer free survival or freedom from symptoms. In certain aspects, a method for treating sarcoma presented herein comprises the administration of Compound 1 or a pharmaceutical composition thereof for a period of time until the severity and/or number of one or more symptoms associated with the sarcoma decreases.

In some aspects, a method for preventing, treating or ameliorating sarcoma in a subject in need thereof comprises the administration of Compound 1 or a pharmaceutical composition thereof for up to 48 weeks. In other aspects, a method for preventing, treating or ameliorating sarcoma in a subject in need thereof comprises the administration of Compound 1 or a pharmaceutical composition thereof for up to 4 weeks, 8 weeks, 12 weeks, 16 weeks, 20 weeks, 24 weeks, 26 weeks (0.5 year), 52 weeks (1 year), 78 weeks (1.5 years), 104 weeks (2 years), or 130 weeks (2.5 years) or more.

In certain aspects, a method for preventing, treating or ameliorating sarcoma in a subject in need thereof comprises the administration of Compound 1 or a pharmaceutical composition thereof for an indefinite period of time. In some aspects, a method for treating sarcoma presented herein comprises the administration of Compound 1 or a pharmaceutical composition thereof for a period of time followed by a period of rest (i.e., a period wherein Compound 1 or a pharmaceutical composition thereof is not administered) before the administration of Compound 1 or a pharmaceutical composition thereof is resumed.

In specific aspects, a method for preventing, treating or ameliorating sarcoma in a subject in need thereof comprises the administration of Compound 1 or a pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, Compound 1 or a pharmaceutical composition thereof may be administered once or twice per week. In a specific aspect of a weekly cycle, Compound 1 or a pharmaceutical composition thereof may be administered twice per week. In a specific aspect of such a weekly cycle, Compound 1 or a pharmaceutical composition thereof may be administered once per day.

In specific aspects, the period of time of administration of Compound 1 or a pharmaceutical composition thereof may be dictated by one or more monitoring parameters, e.g., concentration of certain biomarkers.

In particular aspects, the period of time of administration of Compound 1 or a pharmaceutical composition thereof may be adjusted based on one or more monitoring parameters, e.g., concentration of biomarkers.

In certain aspects, in accordance with a method for preventing, treating or ameliorating sarcoma in a subject in need thereof, Compound 1 or a pharmaceutical composition thereof is administered to a subject in need thereof prior to, concurrently with, or after a meal (e.g., breakfast, lunch, or dinner). In specific aspects, in accordance with the methods for treating sarcoma presented herein, Compound 1 or a pharmaceutical composition thereof is administered to a subject in need thereof in the morning (e.g., between 5 am and 12 pm).

In certain aspects, in accordance with a method for preventing, treating or ameliorating sarcoma in a subject in need thereof, Compound 1 or a pharmaceutical composition thereof is administered to a subject in need thereof at noon (i.e., 12 pm). In particular aspects, in accordance with the methods for treating sarcoma presented herein, Compound 1 or a pharmaceutical composition thereof is administered to a subject in need thereof in the afternoon (e.g., between 12 pm and 5 pm), evening (e.g., between 5 pm and bedtime), and/or before bedtime.

In a specific aspect, a dose of Compound 1 or a pharmaceutical composition thereof is administered to a subject once per day and twice per week.

Combination Therapies

Presented herein are combination therapies for the treatment of sarcoma which involve the administration of Compound 1 or a pharmaceutical composition thereof in combination with one or more additional therapies to a subject in need thereof. In a specific aspect, presented herein are combination therapies for the treatment of sarcoma which involve the administration of an effective amount of Compound 1 or a pharmaceutical composition thereof in combination with an effective amount of another therapy to a subject in need thereof.

As used herein, the term “in combination,” refers, in the context of the administration of Compound 1 or a pharmaceutical composition thereof, to the administration of Compound 1 or a pharmaceutical composition thereof prior to, concurrently with, or subsequent to the administration of one or more additional therapies (e.g., agents, surgery, or radiation) for use in treating sarcoma. The use of the term “in combination” does not restrict the order in which one or more therapeutic agents and one or more additional therapies are administered to a subject. In specific aspects, the interval of time between the administration of Compound 1 or a pharmaceutical composition thereof and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain aspects, Compound 1 or a pharmaceutical composition thereof and one or more additional therapies are administered less than 1 day, 1 week, 2 weeks, 3 weeks, 4 weeks, one month, 2 months, 3 months, 6 months, 1 year, 2 years, or 5 years apart.

In some aspects, the combination therapies provided herein involve administering Compound 1 or a pharmaceutical composition thereof daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain aspects, Compound 1 or a pharmaceutical composition thereof and one or more additional therapies are cyclically administered to a subject. Cycling therapy comprises the administration of Compound 1 or a pharmaceutical composition thereof for a period of time, followed by the administration of one or more additional therapies for a period of time, and repeating this sequential administration. In certain aspects, cycling therapy may also include a period of rest where Compound 1 or a pharmaceutical composition thereof or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an aspect, the number of cycles administered is from 1 to 12 cycles, from 2 to 10 cycles, or from 2 to 8 cycles.

In some aspects, a method for preventing, treating or ameliorating sarcoma in a subject in need thereof comprises administering Compound 1 or a pharmaceutical composition thereof as a single agent for a period of time prior to administering Compound 1 or a pharmaceutical composition thereof in combination with an additional therapy. In certain aspects, the methods for treating sarcoma provided herein comprise administering an additional therapy alone for a period of time prior to administering Compound 1 or a pharmaceutical composition thereof in combination with the additional therapy.

In some aspects, the administration of Compound 1 or a pharmaceutical composition thereof and one or more additional therapies in accordance with the methods presented herein have an additive effect relative the administration of Compound 1 or a pharmaceutical composition thereof or said one or more additional therapies alone. In some aspects, the administration of Compound 1 or a pharmaceutical composition thereof and one or more additional therapies in accordance with the methods presented herein have a synergistic effect relative to the administration of Compound 1 or a pharmaceutical composition thereof or said one or more additional therapies alone.

As used herein, the term “synergistic,” refers to the effect of the administration of Compound 1 or a pharmaceutical composition thereof in combination with one or more additional therapies (e.g., agents), which combination is more effective than the additive effects of any two or more single therapies (e.g., agents).

In a specific aspect, a synergistic effect of a combination therapy permits the use of lower dosages (i.e., sub-optimal doses) of Compound 1 or a pharmaceutical composition thereof or an additional therapy and/or less frequent administration of Compound 1 or a pharmaceutical composition thereof or an additional therapy to a subject.

In certain aspects, the ability to utilize lower dosages of Compound 1 or a pharmaceutical composition thereof or of an additional therapy and/or to administer Compound 1 or a pharmaceutical composition thereof or said additional therapy less frequently reduces the toxicity associated with the administration of Compound 1 or a pharmaceutical composition thereof or of said additional therapy, respectively, to a subject without reducing the efficacy of Compound 1 or a pharmaceutical composition thereof or of said additional therapy, respectively, in the treatment of sarcoma.

In some aspects, a synergistic effect results in improved efficacy of Compound 1 or a pharmaceutical composition thereof and each of said additional therapies in treating sarcoma. In some aspects, a synergistic effect of a combination of Compound 1 or a pharmaceutical composition thereof and one or more additional therapies avoids or reduces adverse or unwanted side effects associated with the use of any single therapy.

The combination of Compound 1 or a pharmaceutical composition thereof and one or more additional therapies can be administered to a subject in the same pharmaceutical composition. Alternatively, Compound 1 or a pharmaceutical composition thereof and one or more additional therapies can be administered concurrently to a subject in separate pharmaceutical compositions. Compound 1 or a pharmaceutical composition thereof and one or more additional therapies can be administered sequentially to a subject in separate pharmaceutical compositions. Compound 1 or a pharmaceutical composition thereof and one or more additional therapies may also be administered to a subject by the same or different routes of administration.

The combination therapies provided herein involve administering to a subject to in need thereof Compound 1 or a pharmaceutical composition thereof in combination with conventional, or known, therapies for treating sarcoma. Other therapies for sarcoma or a condition associated therewith are aimed at controlling or relieving one or more symptoms. Accordingly, in some aspects, the combination therapies provided herein involve administering to a subject to in need thereof a pain reliever, or other therapies aimed at alleviating or controlling one or more symptoms associated with sarcoma or a condition associated therewith.

In one aspect, examples of anti-cancer agents that may be used in combination with Compound 1 or a pharmaceutical composition thereof for treating sarcoma include: a hormonal agent (e.g., aromatase inhibitor, selective estrogen receptor modulator (SERM), and estrogen receptor antagonist), chemotherapeutic agent (e.g., microtubule dissembly blocker, antimetabolite, topisomerase inhibitor, and DNA crosslinker or damaging agent), anti-angiogenic agent (e.g., VEGF antagonist, receptor antagonist, integrin antagonist, vascular targeting agent (VTA)/vascular disrupting agent (VDA)), radiation therapy, and conventional surgery.

In another aspect, non-limiting examples of hormonal agents that may be used in combination with Compound 1 or a pharmaceutical composition thereof for treating sarcoma include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

In another aspect, non-limiting examples of chemotherapeutic agents that may be used in combination with Compound 1 or a pharmaceutical composition thereof for treating cancer include microtubule disassembly blocker, antimetabolite, topoisomerase inhibitor, and DNA crosslinker or damaging agent.

In another aspect, chemotherapeutic agents that are microtubule disassembly blockers include, but are not limited to, taxenes (e.g., paclitaxel (branded/marketed as TAXOL®), docetaxel (branded/marketed as TAXOTERE®), nabPaclitaxel (branded/marketed as ABRAXANE®), larotaxel, ortataxel, and tesetaxel); epothilones (e.g., ixabepilone); and vincalkaloids (e.g., vinorelbine, vinblastine, vindesine, and vincristine (branded/marketed as ONCOVIN®)).

In another aspect, chemotherapeutic agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (branded/marketed as GEMZAR®), cytarabine, decitabine, floxuridine, tegafur); and deoxyribonucleotide antimetabolites (e.g., hydroxyurea).

In another aspect, chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, liposomal doxorubicin, epirubicin, aclarubicin, amrubicin, daunorubicin, idarubicin, pirarubicin, valrubicin, and zorubicin); and anthracenediones (e.g., mitoxantrone, and pixantrone).

In another aspect, chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine (branded/marketed as DTIC-DOME®), temozolomide (branded/marketed as TEMODAR®), altretamine, mitobronitol); and intercalating agents (e.g., actinomycin, bleomycin, mitomycin, and plicamycin).

In another aspect, non-limiting examples of anti-angiogenic agents that may be used in combination with Compound 1 or a pharmaceutical composition thereof for treating sarcoma include VEGF antagonists, receptor antagonists, integrin antagonists (e.g., vitaxin, cilengitide, and S247), and VTAs/VDAs (e.g., fosbretabulin). VEGF antagonists include, but are not to, anti-VEGF antibodies (e.g., bevacizumab (branded/marketed as AVASTIN®) and ranibizumab (branded/marketed as LUCENTIS®)), VEGF traps (e.g., aflibercept), VEGF antisense or siRNA or miRNA, and aptamers (e.g., pegaptanib (branded/marketed as MACUGEN®)). Anti-angiogenic agents that are receptor antagonists include, but are not limited to, antibodies (e.g., ramucirumab) and kinase inhibitors (e.g., sunitinib, sorafenib, cediranib, panzopanib, vandetanib, axitinib, and AG-013958) such as tyrosine kinase inhibitors. Other non-limiting examples of anti-angiogenic agents include ATN-224, anecortave acetate (branded/marketed as RETAANE®), microtubule depolymerization inhibitor such as combretastatin A4 prodrug, and protein or protein fragment such as collagen 18 (endostatin).

In another aspect, non-limiting examples of other therapies that may be administered to a subject in combination with Compound 1 or a pharmaceutical composition thereof for treating sarcoma include:
(1) a statin such as lovostatin (e.g., branded/marketed as MEVACOR®);
(2) an mTOR inhibitor such as sirolimus which is also known as Rapamycin (e.g., branded/marketed as RAPAMUNE®), temsirolimus (e.g., branded/marketed as TORISEL®), evorolimus (e.g., branded/marketed as AFINITOR®), and deforolimus;
(3) a farnesyltransferase inhibitor agent such as tipifarnib (e.g., branded/marketed as ZARNESTRA®);
(4) an antifibrotic agent such as pirfenidone;
(5) a pegylated interferon such as PEG-interferon alfa-2b;
(6) a CNS stimulant such as methylphenidate (branded/marketed as RITALIN®);
(7) a HER-2 antagonist such as anti-HER-2 antibody (e.g., trastuzumab) and kinase inhibitor (e.g., lapatinib);
(8) an IGF-1 antagonist such as an anti-IGF-1 antibody (e.g., AVE1642 and IMC-A11) or an IGF-1 kinase inhibitor;
(9) EGFR/HER-1 antagonist such as an anti-EGFR antibody (e.g., cetuximab, panitumamab) or EGFR kinase inhibitor (e.g., erlotinib (e.g., branded/marketed as TARCEVA®), gefitinib);
(10) SRC antagonist such as bosutinib;
(11) cyclin dependent kinase (CDK) inhibitor such as seliciclib;
(12) Janus kinase 2 inhibitor such as lestaurtinib;
(13) proteasome inhibitor such as bortezomib;
(14) phosphodiesterase inhibitor such as anagrelide;
(15) inosine monophosphate dehydrogenase inhibitor such as tiazofurine;
(16) lipoxygenase inhibitor such as masoprocol;
(17) endothelin antagonist;
(18) retinoid receptor antagonist such as tretinoin or alitretinoin;
(19) immune modulator such as lenalidomide, pomalidomide, or thalidomide (e.g., branded/marketed as THALIDOMID®);
(20) kinase (eg, tyrosine kinase) inhibitor such as imatinib (e.g., branded/marketed as GLEEVEC®), dasatinib, erlotinib, nilotinib, gefitinib, sorafenib, sunitinib (e.g., branded/marketed as SUTENT®), lapatinib, AEE788, or TG100801;
(21) non-steroidal anti-inflammatory agent such as celecoxib (branded/marketed as CELEBREX®);
(22) human granulocyte colony-stimulating factor (G-CSF) such as filgrastim (branded/marketed as NEUPOGEN®);
(23) folinic acid or leucovorin calcium;
(24) integrin antagonist such as an integrin α5β1-antagonist (e.g., JSM6427);
(25) nuclear factor kappa beta (NF-κβ) antagonist such as OT-551, which is also an anti-oxidant;
(26) hedgehog inhibitor such as CUR61414, cyclopamine, GDC-0449, or anti-hedgehog antibody;
(27) histone deacetylase (HDAC) inhibitor such as SAHA (also known as vorinostat (branded/marketed as ZOLINZA®)), PCI-24781, SB939, CHR-3996, CRA-024781, ITF2357, JNJ-26481585, or PCI-24781;
(28) retinoid such as isotretinoin (e.g., branded/marketed as ACCUTANE®);
(29) hepatocyte growth factor/scatter factor (HGF/SF) antagonist such as HGF/SF monoclonal antibody (e.g., AMG 102);
(30) synthetic chemical such as antineoplaston;
(31) anti-diabetic such as rosiglitazone maleate (e.g., branded/marketed as AVANDIA®);
(32) antimalarial and amebicidal drug such as chloroquine (e.g., branded/marketed as ARALEN®);
(33) synthetic bradykinin such as RMP-7;
(34) platelet-derived growth factor receptor inhibitor such as SU-101;
(35) receptor tyrosine kinase inhibitorsof Flk-1/KDR/VEGFR2, FGFR1 and PDGFR beta such as SU5416 and SU6668;
(36) anti-inflammatory agent such as sulfasalazine (e.g., branded/marketed as AZULFIDINE®); and
(37) TGF-beta antisense therapy.

In another aspect, non-limiting examples of other therapies that may be administered to a subject in combination with Compound 1 or a pharmaceutical composition thereof for treating sarcoma include: a synthetic nonapeptide analog of naturally occurring gonadotropin releasing hormone such as leuprolide acetate (branded/marketed as LUPRON®); a nonsteroidal, anti-androgen such as flutamide (branded/marketed as EULEXIN®) or nilutamide (branded/marketed as NILANDRON®); a non-steroidal androgen receptor inhibitor such as bicalutamide (branded/marketed as CASODEX®); steroid hormone such as progesterone; anti-fungal agent such as Ketoconazole (branded/marketed as NIZORAL®); glucocorticoid such as prednisone; estramustine phosphate sodium (branded/marketed as EMCYT®); and bisphosphonate such as pamidronate, alendronate, and risedronate.

In another aspect, examples of therapies that may be used in combination with Compound 1 or a pharmaceutical composition thereof for treating sarcoma include, but are not limited to, agents associated with cancer immunotherapy (e.g., cytokines, interleukins, and cancer vaccines).

In one aspect, chemotherapeutic combination therapies include administration of Compound 1 in combination with at least one chemotherapeutic agent, wherein the chemotherapeutic agent is selected from the group consisting of DTIC-DOME® (dacarbazine), TAXOTERE® (docetaxel), ADRIAMYCIN® or RUBEX® (doxorubicin), DOXIL® (liposomal doxorubicin), gemcitabine, epirubicin, eribulin, ifosfamide, temozolomide, trabectedin, and ONCOVIN® (vincristine).

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, gemcitabine, epirubicin, eribulin, ifosfamide, temozolomide, trabectedin, and vincristine.

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, gemcitabine, and vincristine.

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, and vincristine.

In certain aspects, Compound 1 or a pharmaceutical composition thereof is not used in combination with a drug that is primarily metabolized by CYP2D6 (such as an antidepressant (e.g, a atricyclic antidepressant, a selective serotonin reuptake inhibitor, and the like), an antipsychotic, a beta-adrenergic receptor blocker, or certain types of anti-arrhythmics) to treat sarcoma.

In another aspect, combination therapies provided herein for treating sarcoma may comprise administering Compound 1 or a pharmaceutical composition thereof in combination with at least one or more agents used to treat and/or manage a side effect, such as, bleeding (usually transient, low-grade epistaxis), subungual hemorrhage, hemorrhagic cystitis (bleeding and irritation of the bladder), arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, myelodysplastic syndromes, myelosuppression, fatigue, hypothyroidism, QT interval prolongation, or heart failure.

In another aspect, examples of agents alleviating side-effects associated with sarcoma that can be used as therapies in combination with Compound 1 or a pharmaceutical composition thereof, include, but are not limited to: antiemetics, e.g., ondansetron hydrochloride (branded/marketed as ZOFRAN®), granisetron hydrochloride (branded/marketed as KYTRIL®), lorazepam (branded/marketed as ATIVAN®) and dexamethasone (branded/marketed as DECADRON®).

In another aspect, examples of agents alleviating side-effects associated with sarcoma chemotherapeutic agent that can be used as therapies in combination with Compound 1 or a pharmaceutical composition thereof, include, but are not limited to: antibleeding agents, e.g., mesna (branded/marketed as Mesnex®).

In another aspect, treatment of sarcoma may include surgery or radiation therapy.

In one aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, gemcitabine, epirubicin, eribulin, ifosfamide, temozolomide, trabectedin, and vincristine.

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, gemcitabine, and vincristine.

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin, and vincristine.

In another aspect, a chemotherapeutic combination therapy may include administration of Compound 1 in combination with dacarbazine and doxorubicin, wherein the combination is administered tiw (once every three weeks), with dacarbazine at 250 mg/m²/day IV continuous infusion for five days (equivalent to 800-1000 mg/m² IV every 3 wk) and doxorubicin at 15 mg/m²/day IV continuous infusion for days 1-4.

In another aspect, a chemotherapeutic combination therapy may include administration of Compound 1 in combination with doxorubicin, ifosfamide, and dacarbazine, wherein the combination is administered tiw (once every three weeks), doxorubicin at 20 mg/m²/day IV continuous infusion for three days, ifosfamide 2.5 g/m²/day IV continuous infusion for three days, and dacarbazine at 300 mg/m²/day IV continuous infusion for three days.

In one aspect, the effective amount of Compound 1 and the effective amount of the chemotherapeutic agent when administered in combination with each other are reduced or administered less frequently compared to regimens tested and known in the art.

In another aspect, chemotherapeutic combination therapies include administration of Compound 1 in combination with at least one chemotherapeutic agent, wherein the chemotherapeutic agent is selected from the group consisting of dacarbazine, docetaxel, doxorubicin, liposomal doxorubicin and vincristine.

Kits

Provided herein is a pharmaceutical pack or kit comprising one or more containers filled with Compound 1 or a pharmaceutical composition thereof. Additionally, one or more other therapies useful for the treatment of sarcoma, or other relevant agents can also be included in the pharmaceutical pack or kit. Also provided herein is a pharmaceutical pack or kit comprising one or more containers filled with one or more of the ingredients of the pharmaceutical compositions described herein. Optionally associated with such kits can be a notice in the form prescribed by a governmental agency regulating the manufacture, use or sale of pharmaceuticals or biological products, which notice reflects approval by the agency of manufacture, use or sale for human administration.

### BIOLOGICAL EXAMPLES

Compound 1 was tested for usefulness in affecting sarcoma proliferation using a comprehensive set of in vitro and in vivo models.

Background

Leiomyosarcoma accounts for 5-10% of soft tissue sarcomas. Typically, leiomyosarcomas have a complex karyotype and are associated with p53 mutations (Yang J, Du X, Chen K, Ylipaa A, Lazar A J, Trent J, Lev D, Pollock R, Hao X, Hunt K, Zhang W. Genetic aberrations in soft tissue leiomyosarcoma. Cancer Lett. 2009 Mar. 8; 275(1):1-8). The cell line, SK-LMS-1 has a complex karyotype and a mutated p53 gene. Chemotherapy with doxorubicin-based drug regimens such as a combination of doxorubicin and ifosfamide is the standard of care. However, these regimens usually result in cardiotoxicity, rapid development of resistance, and no significant survival advantage. In a previous study utilizing the SK-LMS-1 model, Compound 1 (Cpd 1) was tested at a constant dose (12.5 mg/kg biw) in combination with varying doses/regimens of doxorubicin and gemcitabine. Cpd 1 and gemcitabine were more effective than either agent as a monotherapy. Doxorubicin was not active as a monotherapy or in combination with Cpd 1. Here, SK-LMS-1 cells were passaged for a more extended time in vivo relative to the prior study generating a more aggressive tumor model. This study tested the efficacy of Cpd 1 alone and in combination with the chemotherapeutic agents Doxil and DTIC in the treatment of mice bearing SK-LMS-1 human leiomyosarcoma tumors.

### Example 1

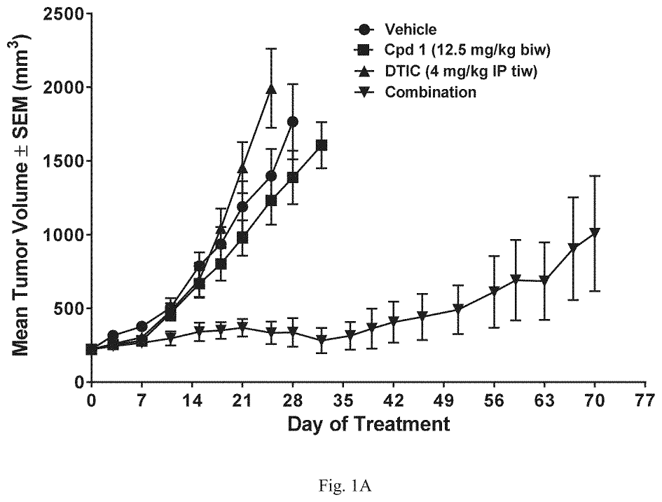

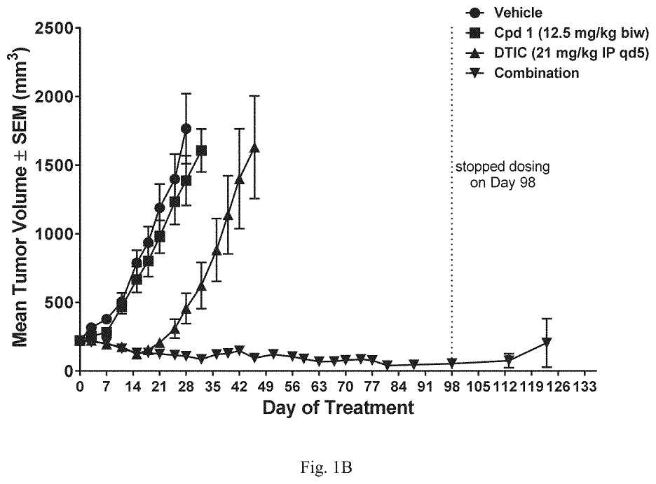

As shown in Table 1, a combination of Compound 1 and dacarbazine (DTIC) was tested in an SK-LMS-1 leiomyosarcoma mouse model. As further shown in FIG. 1A and FIG. 1B, the combination of Compound 1 (12.5 mg/kg, PO, biw) and low dose DTIC (4 mg/kg, IP, tiw) and Compound 1 (12.5 mg/kg, PO, biw) and high dose DTIC (21 mg/kg, IP, qd5) were tested and compared with results from administration of Compound 1 alone, low dose DTIC alone, high dose DTIC alone and vehicle, where biw represents dose administration two times per week; where tiw represents dose administration three times per week; where qd5 represents dose administration every day for five days; where IP represents intraperitoneal administration; where IV represents intravenous administration; and, where PO represents per os (oral gavage dosing). As shown in each Figure, the combination resulted in a synergistic reduction in mean tumor volume. Comparison of FIG. 1A and FIG. 1B show a dose response reduction in mean tumor volume after treatment with a combination of Compound 1 (12.5 mg/kg, PO, biw) and DTIC (4 mg/kg, IP, tiw) (see, FIG. 1A) compared to treatment with a combination of Compound 1 (12.5 mg/kg, PO, biw) and DTIC (21 mg/kg, IP, qd5) (see, FIG. 1B). Table 1 further shows, for the mice tested (N) in each Treatment Group, the percent reduction in tumor volume at Day 28(%), the median time for tumor volume for individual mice to reach 1000 mm³ (Days) and corresponding Interaction Coefficient (IC) for each, where * represents p<0.05 (ANOVA, multiple comparisons vs. vehicle). A negative coefficient indicates greater inhibition in the combination group than expected based on the activity of the monotherapies. The more negative the coefficient, the greater the activity of the combination.

TABLE 1

| | | Dose | | Dose (mg/kg), | | | | | |
|---|---|---|---|---|---|---|---|---|---|
| | | (mg/kg), | | Route, | | | | | |
| Group | Treatment | Regimen | Agent | Regimen | N | % | IC | Days | IC |
| 1 | Vehicle | 0, biw | None | None | 8 | 0 | | 18 | |
| 2 | Cpd 1 | 12.5, biw | None | None | 8 | 21 | | 23 | |
| 3 | Vehicle | 0, biw | DTIC | 4 mg/kg IP tiw | 8 | 0 | −0.33 | 19 | −0.41 |
| 4 | Cpd 1 | 12.5, biw | DTIC | | 8 | 81* | | 55* | |
| 5 | Vehicle | 0, biw | DTIC | 21 mg/kg IP | 8 | 74* | −0.029 | 35 | −0.29 |
| 6 | Cpd 1 | 12.5, biw | DTIC | qd5 | 8 | 94* | | > 165* | |

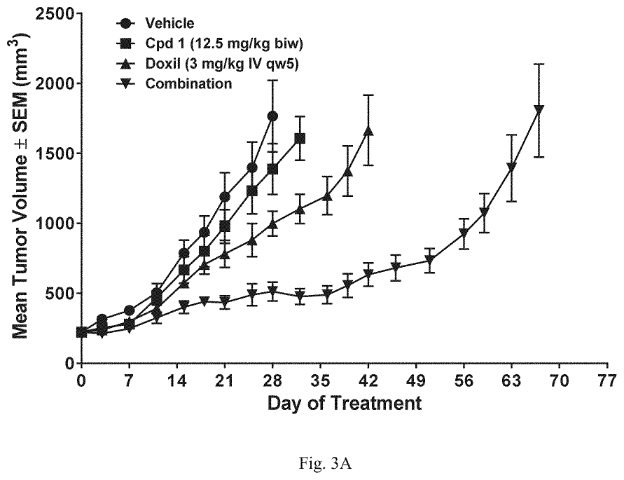

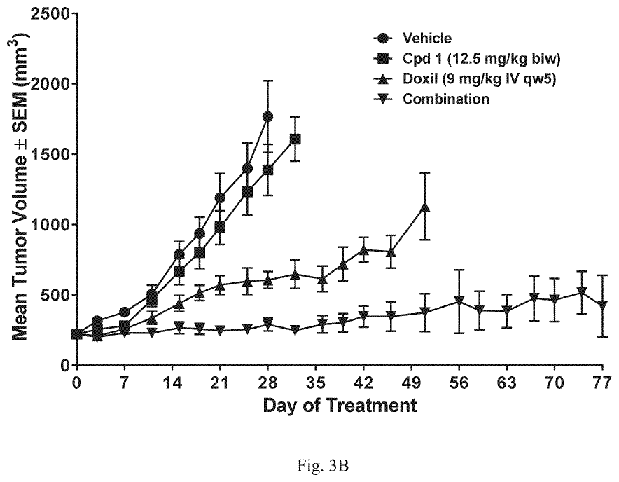

Tumors were ˜222 mm3 in volume when dosing was initiated. As shown in FIGS. 1A and 1B, 3A and 3, tumors in mice dosed only with vehicle reached an average volume of 1766 mm³ by Day 28. On Day 28, most of the mice dosed with vehicle were euthanized due to the large tumors (7/8; in one mouse the tumor did not grow). The time for the mean tumor volume to reach 1000 mm³ in mice dosed with vehicle was 19 days. In mice treated with Cpd 1, tumor growth was modestly delayed, and reached a mean volume of 1388 mm3 on Day 28 and 1607 mm³ on Day 32 at which time all of the mice in the group had large tumors and were euthanized. The Interaction Coefficients assessing whether the measured effect is greater than the predicted effect are summarized in Table 1 for each combination. Interaction coefficients were determined using the AUCs of the Day 0 to Day 28 tumor volume vs day curves. To take into account the data after Day 28, interaction coefficients were calculated for the median time to reach 1000 mm³. A negative interaction coefficient indicates synergy.

### Example 1 Results

Cpd 1 (12.5 mg/kg, biw) modestly delayed tumor growth as a monotherapy. DTIC (21 mg/kg, IP, qd5) was more effective at the higher dose as a monotherapy than Cpd 1, delaying tumor growth. DITC (4 mg/kg, IP, tiw) had little efficacy at the lower dose as a monotherapy.

The combination of Cpd 1 with DITC at the lower dose was more effective than Cpd 1 alone or DTIC alone at the lower dose. This was unexpected because DTIC demonstrated little efficacy as a monotherapy at the lower dose. The combination of Cpd 1 with DTIC at the higher dose was more effective than Cpd 1 alone or DTIC alone at the higher dose.

FIG. 1A shows the mean tumor volumes over time for the vehicle, Cpd 1, DTIC (4 mg/kg IP tiw), or the combination of Cpd 1 and DTIC (4 mg/kg IP tiw). DTIC had no activity at a dose/regimen of 4 mg/kg IP tiw. Surprisingly, the combination of Cpd 1 and DTIC was much more effective than either agent alone. Three mice remained on study through Day 136, and two mice were on study through Day 150 at which time they were taken off study. On Day 150, one of the two tumors was too small to measure.

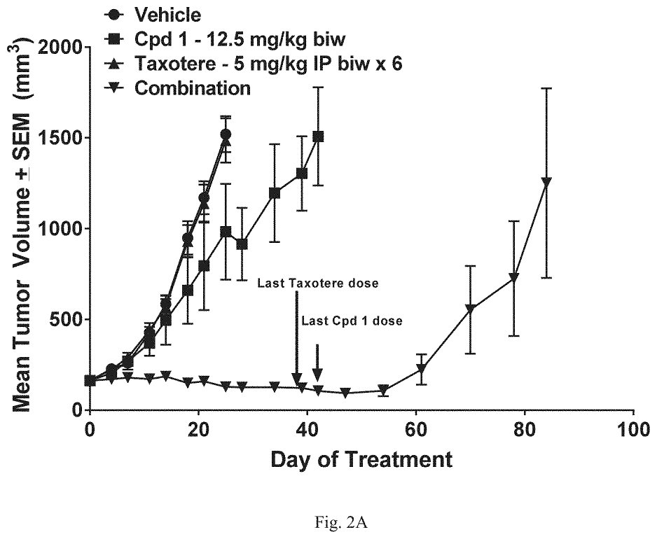

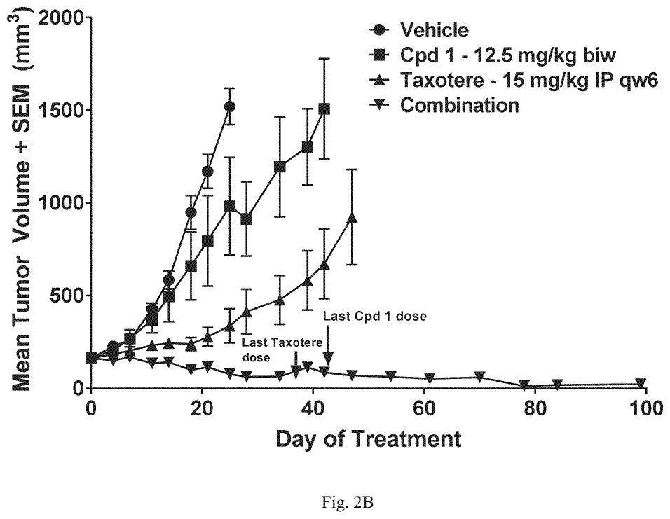

FIG. 2B shows the mean tumor volumes with time for the vehicle, Cpd 1 alone, DTIC alone (4 mg/kg IP tiw) and DTIC alone (21 mg/kg IP qd5), or the combination of Cpd 1 and DTIC (4 mg/kg IP tiw) or Cpd 1 and DTIC (21 mg/kg IP qd5). Using this dose and regimen of DTIC, DTIC was more effective than Cpd 1 and completely prevented tumor growth through Day 18, although after Day 21 rapidly growing tumors escaped. The efficacy of Cpd 1 in combination DTIC (21 mg/kg IP qd5) was much greater than that of either agent alone, with near complete inhibition of tumor growth at Day 98. After Day 98, mice were no longer dosed with Cpd 1. From Day 98 to Day 150, when mice were taken off study, in the 8 mice remaining on study, the tumors in three grew larger but tumors in five did not. One mouse was taken off study with a large tumor on Day 123. At Day 165, when the remaining 7 mice were taken off study, 5 mice had tumors too small to measure and the other two mice had tumors that were palpable, but smaller than 100 mm³.

For both doses, the effect of the combination of Cpd 1 and DTIC was greater than either therapy alone (the Interaction Coefficient was negative; Table 1). Greater additivity was observed using a dose of 21 mg/kg qd5 than 4 mg/kg tiw.

### Example 2

As shown in Table 2, a combination of Compound 1 and docetaxel was tested in an SK-UT-1 leiomyosarcoma mouse model. As further shown in FIG. 2A and FIG. 2B, the combination of Compound 1 (12.5 mg/kg PO biw) and low dose docetaxel (5 mg/kg IP biw5.5 for 11 doses total), and Compound 1 (12.5 mg/kg PO biw) and high dose docetaxel (15 mg/kg IP qw6)(six doses total) were tested and compared with results from administration of Compound 1 alone, low dose docetaxel alone, high dose docetaxel alone and vehicle, where biw represents dose administration two times per week; and, where qw6 represents dose administration once per week for six weeks; where IP represents intraperitoneal administration; and, where PO represents per os (oral gavage dosing). As shown in each Figure, the combination resulted in a synergistic reduction in mean tumor volume. Comparison of FIG. 2A and FIG. 2B show a dose dependent suppression of mean tumor growth after treatment with a combination of Compound 1 (12.5 mg/kg PO biw) and docetaxel (5 mg/kg IP biw for 11 doses total) (see, FIG. 2A) compared to treatment with a combination of Compound 1 (12.5 mg/kg PO biw) and docetaxel (15 mg/kg IP qw6) (see, FIG. 2B). Table 2 further shows, for the mice tested (N) in each Treatment Group, the percent reduction in tumor volume at Day 28(%), the median time for tumor volume for individual mice to reach 1000 mm³ (Days) and corresponding Interaction Coefficient (IC) for each, where * represents p<0.05 (ANOVA, multiple comparisons vs. vehicle). A negative coefficient indicates greater inhibition in the combination group than expected based on the activity of the monotherapies. The more negative the coefficient, the greater the activity of the combination.

TABLE 2

| | | Dose | | | | | | | |
|---|---|---|---|---|---|---|---|---|---|
| | | (mg/kg), | | Dose (mg/kg), | | | | | |
| Group | Treatment | Regimen | Agent | Route, Regimen | N | % | IC | Days | IC |
| 1 | Vehicle | 0, biw6 | None | None | 10 | 0.0 | | 18.5 | |
| 2 | Cpd 1 | 12.5, biw6 | None | None | 10 | 35.4* | | 31.2 | |
| 3 | Vehicle | 0, biw6 | docetaxel | 5 mg/kg IP | 10 | 2.27 | −0.49 | 18.9 | −0.45 |
| 4 | Cpd 1 | 12.5, biw6 | docetaxel | biw5.5 | 10 | 91.6* | | >145* | |
| 5 | Vehicle | 0, biw6 | docetaxel | 15 mg/kg IP | 10 | 77.8* | −1.1 | 50.1* | −0.09 |
| 6 | Cpd 1 | 12.5, biw6 | docetaxel | qw6 | 10 | 94.9* | | >145* | |

The average tumor volume was ˜163 mm³ when dosing was initiated. As shown in FIGS. 2A and 2B, tumors in mice dosed only with vehicle (Group 1) reached an average volume of 1520 mm³ on Day 25. On Day 25, mice dosed with vehicle were euthanized due to the large tumors. The time for the mean tumor volume to reach 1000 mm³ in mice dosed with vehicle was 18.5 days. In mice treated with Cpd 1 (Group 2), tumor growth was delayed, and reached a mean volume of 983 mm³ on Day 25 (35% inhibition vs vehicle; p<0.05, analysis of covariance (ANOVA), multiple comparisons vs vehicle) and 1508 mm³ on Day 42 at which time all of the mice in the group had large tumors and were euthanized. In mice treated with docetaxel, tumor growth was delayed at a dose of 15 mg/kg intraperitoneal (IP) once per week for 6 weeks (QW6; Group 5) but not at 5 mg/kg IP BIW×5.5 (11 doses total; Group 3).

This study utilized SK-UT-1 leiomyosarcoma tumor cells implanted into the flank of athymic nude mice. In this model, Cpd 1 at a dose of 12.5 mg/kg BIW6 demonstrated efficacy as monotherapy, reducing tumor growth by ˜35% at Day 25 and delaying the median time for tumor growth to reach 1000 mm³ by ˜1.7-fold. As a monotherapy, high dose docetaxel (15 mg/kg IP QW6), at a tumor size of 1486 mm³, showed 78% inhibition at Day 25 (when mice were taken off study), having a p<0.05 by analysis of covariance (ANOVA, multiple comparisons vs vehicle). Low dose docetaxel (5 mg/kg IP BIW5.5) had no efficacy as a monotherapy but the combination of Cpd 1 and low dose docetaxel significantly delayed tumor growth. At Day 25, the mean tumor size in mice dosed with the Cpd 1 and low dose docetaxel combination was 128 mm³, showing 92% inhibition vs vehicle, having a p<0.05 (ANOVA, multiple comparisons vs vehicle) at a tumor size of 1251 mm³ on Day 84. The combination of Cpd 1 and high dose docetaxel was more effective than either agent alone. At Day 25, the mean tumor size in mice dosed with the combination was 77 mm³, showing 95% inhibition vs vehicle (p<0.05, ANOVA, multiple comparisons vs vehicle). At Day 145, 8/10 mice were still on study, with all 8 having tumors that were too small to measure (<50 mm3).

### Example 2 Results

Cpd 1 at 12.5 mg/kg BIW6 modestly delayed tumor growth as a monotherapy. Low dose docetaxel had no efficacy as a monotherapy at 5 mg/kg IP BIW5.5 (for 11 doses), but high dose docetaxel at 15 mg/kg IP QW6 was more effective than Cpd 1.

The combination of either low or high dose docetaxel and Cpd 1 was more effective than either drug as monotherapy.

A higher rate of morbidity/mortality was seen with the combination of Cpd 1 and docetaxel. With the combination of low dose docetaxel (5 mg/kg IP BIW5.5) and Cpd 1, 5/10 mice were taken off as moribund or euthanized before the tumor had reached 1000 mm³. With the combination of high dose docetaxel (15 mg/kg IP QW6) and Cpd 1, 2/10 mice were taken off as moribund or euthanized before the tumor had reached 1000 mm³.

FIG. 2A shows the mean tumor volumes over time for the vehicle, Cpd 1 alone, low dose docetaxel alone (5 mg/kg IP BIW5.5; dosing twice per week for 5½ weeks, 11 total doses), and the combination of Cpd 1 and low dose docetaxel. The low dose docetaxel alone had no efficacy as a monotherapy at a dose of 5 mg/kg IP BIW5.5 but the combination of Cpd 1 and low dose docetaxel significantly delayed tumor growth. At Day 25, the mean tumor size in mice dosed with the combination was 128 mm³, 92% inhibition vs vehicle (p<0.05, ANOVA, multiple comparisons vs vehicle) and 1251 mm³ on Day 84.

FIG. 2B shows the mean tumor volumes over time for the vehicle, Cpd 1, high dose docetaxel (15 mg/kg IP QW6), or the combination of Cpd 1 and high dose docetaxel. As monotherapy, high dose docetaxel, for tumors at 1486 mm³, showed 78% inhibition at Day 25 at which time the mice were taken off study (p<0.05, ANOVA, multiple comparisons vs vehicle). The combination of Cpd 1 and high dose docetaxel was more effective than either therapy alone. At Day 25, the mean tumor size in mice dosed with the combination was 77 mm³, showing 95% inhibition vs vehicle (p<0.05, ANOVA, multiple comparisons vs vehicle). At Day 145, 8/10 of the mice were still on study, and all 8 had tumors that were too small to measure (<50 mm³).

To compare tumor growth, the area under the curve comparing tumor volume vs day for each individual mouse was calculated and then averaged for each group. As shown in FIG. 1, greater efficacy was seen with the combination of Cpd 1 and docetaxel relative to monotherapy treatment.

Interaction coefficients assessing if the measured effect is greater than the predicted effect are shown in Table 2. Interaction coefficients were determined using the AUCs of the Day 0 to Day 25 tumor volume vs day curves. To take into account the data after Day 25, interaction coefficients were also calculated for the median time to reach 1000 mm3. A negative interaction coefficient indicates synergy.

### Example 3

As shown in Table 3, a combination of Compound 1 and doxil was tested in an SK-LMS-1 leiomyosarcoma mouse model. As further shown in FIG. 3A and FIG. 3B, the combination of Compound 1 (12.5 mg/kg, PO, biw), low dose doxil (3 mg/kg, IV, qw5) and high dose doxil (9 mg/kg, IV, qw5) was tested and compared with results from administration of Compound 1 alone, doxil alone and vehicle, where biw represents dose administration two times per week; and, where qw5 represents dose administration once per week for five weeks; where IV represents intravenous administration; and, where PO represents per os (oral gavage dosing). As shown in each Figure, the combination resulted in a synergistic reduction in mean tumor volume. Comparison of FIG. 3A and FIG. 3B show a dose dependent suppression of mean tumor growth after treatment with a combination of Compound 1 (12.5 mg/kg PO biw) and low dose doxil (3 mg/kg IV qw5) (see, FIG. 3A) compared to treatment with a combination of Compound 1 (12.5 mg/kg PO biw) and high dose doxil (9 mg/kg IV qw5) (see, FIG. 3B). Table 1 further shows, for the mice tested (N) in each Treatment Group, the percent reduction in tumor volume at Day 28(%), the median time for tumor volume for individual mice to reach 1000 mm³ (Days) and corresponding the Interaction Coefficient (IC) for each, where * represents p<0.05 (ANOVA, multiple comparisons vs. vehicle). A negative coefficient indicates greater inhibition in the combination group than expected based on the activity of the monotherapies. The more negative the coefficient, the greater the activity of the combination.

TABLE 3

| | | | | Dose | | | | | |
|---|---|---|---|---|---|---|---|---|---|
| | | | | (mg/kg), | | | | | |
| | | Dose (mg/kg), | | Route, | | | | | |
| Group | Treatment | Regimen | Agent | Regimen | N | % | IC | Days | IC |
| 1 | Vehicle | 0, biw | None | None | 8 | 0 | | 18 | |
| 2 | Cpd 1 | 12.5, biw | None | None | 8 | 21 | | 23 | |
| 3 | Vehicle | 0, biw | Doxil | 3 mg/kg IV | 8 | 43* | −0.13 | 30 | −0.15 |
| 4 | Cpd 1 | 12.5, biw | Doxil | qw5 | 8 | 71* | | 56* | |
| 5 | Vehicle | 0, biw | Doxil | 9 mg/kg IV | 8 | 66* | −0.13 | 57* | −0.096 |
| 6 | Cpd 1 | 12.5, biw | Doxil | on day 0, 9, | 8 | 84* | | 119* | |
| | | | | 15, 21 and 28 | | | | | |

Tumors were ˜222 mm3 in volume when dosing was initiated. As shown in FIGS. 3A and 3B, tumors in mice dosed only with vehicle reached an average volume of 1766 mm³ by Day 28. On Day 28, most of the mice dosed with vehicle were euthanized due to the large tumors (7/8; in one mouse the tumor did not grow). The time for the mean tumor volume to reach 1000 mm³ in mice dosed with vehicle was 19 days. In mice treated with Cpd 1, tumor growth was modestly delayed, and reached a mean volume of 1388 mm3 on Day 28 and 1607 mm³ on Day 32 at which time all of the mice in the group had large tumors and were euthanized. The Interaction Coefficients assessing whether the measured effect is greater than the predicted effect are summarized in Table 3 for each combination. Interaction coefficients were determined using the AUCs of the Day 0 to Day 28 tumor volume vs day curves. To take into account the data after Day 28, interaction coefficients were calculated for the median time to reach 1000 mm³. A negative interaction coefficient indicates synergy.

### Example 3 Results

Cpd 1 (12.5 mg/kg, biw) modestly delayed tumor growth as a monotherapy. At both the low dose and high dose doxil was more effective as a monotherapy than Cpd 1, delaying tumor growth. However, the high dose doxil regimen was poorly tolerated.

The combination of Cpd 1 with doxil at the lower dose was more effective than Cpd 1 alone or doxil alone at the lower dose. The combination of Cpd 1 with doxil at the higher dose was more effective than both Cpd 1 alone or higher dose doxil alone. However, the combination of Cpd 1 with high dose doxil was poorly tolerated.

FIG. 3A shows the mean tumor volumes over time for the vehicle, Cpd 1, doxil (3 mg/kg IV qw5), or the combination of Cpd 1 and doxil (3 mg/kg IV qw5).

FIG. 3B shows the mean tumor volumes over time for the vehicle, Cpd 1 alone, doxil alone (9 mg/kg IV on Days 0, 9, 15, 21 and 28), or the combination of Cpd 1 and doxil (9 mg/kg IV). Doxil delayed the growth of SK-LMS-1 as monotherapy more effectively than did Cpd 1 and was more effective at 9 mg/kg IV (Day 0, 9, 15, 21 and 28) than at 3 mg/kg IV qw5. Although Cpd 1 had only limited efficacy as monotherapy, Cpd 1 enhanced the efficacy of doxil using both regimens (the Interaction Coefficient was negative indicating synergy).

### Example 4 and Results

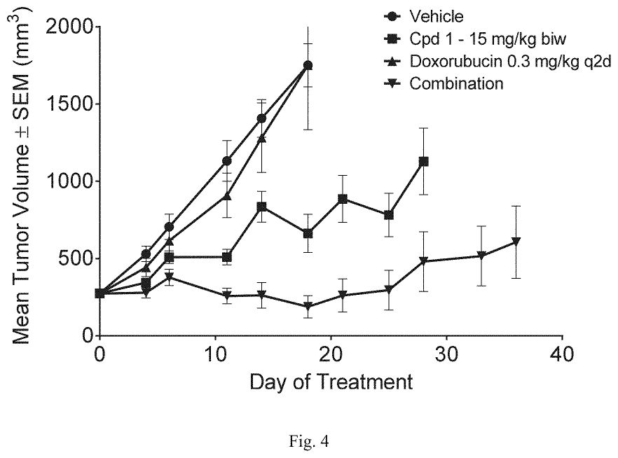

A combination of Compound 1 and doxorubicin was tested in a HT1080 fibrosarcoma mouse model. As shown in FIG. 4, the combination of Compound 1 (15 mg/kg PO biw) and doxorubucin (0.3 mg/kg IP q2d) was tested and resulted in a synergistic reduction in mean tumor volume when compared with results from administration of Compound 1 alone, doxorubucin alone and vehicle, where biw represents dose administration two times per week; and, where q2d represents dose administration once every two days.

### Example 5 and Results

Compound 1 was administered as a first-line therapy in NCT02404480, a Phase 1a clinical trial designed to evaluate safety and PK profile for use of Compound 1 in patients with advanced solid tumor (AST) cancers. As shown in FIG. 5 for 31 evaluable patients, 2/31 patients had a mixed/partial response and 5/31 patients resulted in stable disease.

### Example 6 and Results

The PK profile (as measured by AUC_{Last} and Cₘₐₓ) from the NCT02404480 clinical trial shown in FIG. 6 indicates that, for a target AUC_{Last} of 13.1 ng-hr/mL and target Cₘₐₓ of 679 ng/mL, an effective dose of 2.6 mg/kg of Compound 1 was selected to be the phase II clinical trial dose.

### Example 7 and Results

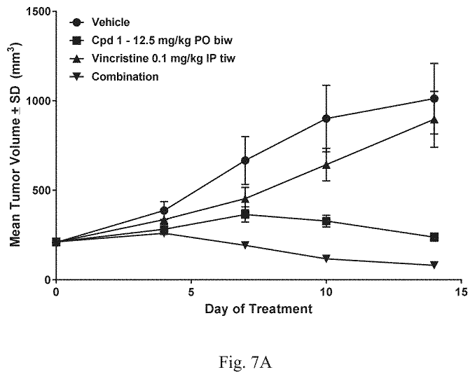

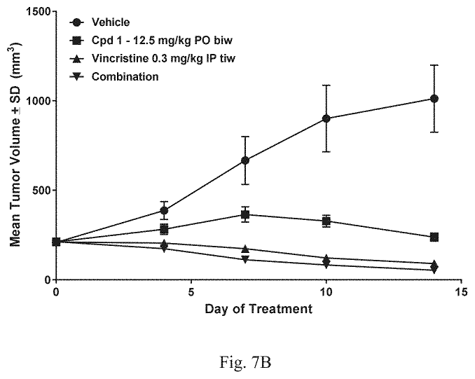

A combination of Compound 1 and vincristine was tested in an HT1080 fibrosarcoma xenograft mouse model. As shown in FIG. 7A and FIG. 7B, the combination of Compound 1 (12.5 mg/kg PO biw) and vincristine (0.1 mg/kg IP tiw) was tested and compared with results from administration of Compound 1 alone, vincristine alone and vehicle, where biw represents dose administration two times per week; and, where tiw represents dose administration three times per week. Comparison of FIG. 7A and FIG. 7B show for each agent a dose dependent decrease in mean tumor growth after treatment with a combination of Compound 1 (12.5 mg/kg PO biw) and vincristine (0.1 mg/kg IP tiw) (see, FIG. 7A) compared to treatment with a combination of Compound 1 (12.5 mg/kg PO biw) and vincristine (0.3 mg/kg IP tiw) (see, FIG. 7B).

As shown herein, Compound 1 has demonstrated pre-clinical in vivo activity as a combination therapy with standard-of-care chemotherapeutic agents for the treatment of sarcoma, showing synergistic and additive activity when administered in combination with at least one such chemotherapeutic agent. These results strongly demonstrate the potential clinical therapeutic utility for combinations of Compound 1 with at least one chemotherapeutic agent for the treatment of sarcoma.

### REFERENCES

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or aspects described herein. It is intended that the appended claims be interpreted to include all such equivalents.

What is claimed is:

1. A method for treating sarcoma in a subject in need thereof comprising, administering to the subject an effective amount of 5-fluoro-2-(6-fluoro-2-methyl-1H-benzo[d]imidazol-1-yl)-N⁴-[4-(trifluoromethyl)phenyl]pyrimidine-4,6-diamine, having the structure of Compound 1: 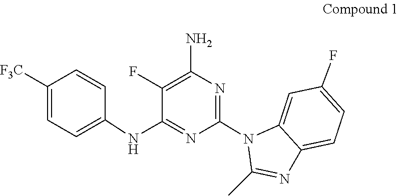 [SMILES: Cc1nc2ccc(F)cc2n1-c1nc(N)c(F)c(Nc2ccc(C(F)(F)F)cc2)n1]
or a pharmaceutically acceptable salt or pharmaceutical composition thereof.

2. The method of claim 1 comprising, administering to the subject an effective amount of Compound 1 or a pharmaceutically acceptable salt or pharmaceutical composition thereof in combination with an effective amount of at least one chemotherapeutic agent.

3. The method of claim 1 wherein, the effective amount is a dose selected from the group consisting of about 100 mg, about 125 mg, about 150 mg, about 175 mg, about 200 mg, about 225 mg, about 250 mg, about 275 mg, and about 300 mg, administered orally twice per week.

4. The method of claim 3 wherein, the effective amount is a dose selected from the group consisting of about 125 mg, about 150 mg, about 175 mg, about 225 mg, about 250 mg, about 275 mg, and about 300 mg, administered orally twice per week.

5. The method of claim 2 wherein, the at least one chemotherapeutic agent is selected from the group consisting of dacarbazine, docetaxel, doxorubicin, liposomal doxorubicin, gemcitabine, epirubicin, eribulin, ifosfamide, temozolomide, trabectedin, and vincristine.

6. The method of claim 5 wherein, the at least one chemotherapeutic agent is selected from the group consisting of dacarbazine, docetaxel, doxorubicin, liposomal doxorubicin, and vincristine.

7. The method of claim 6, wherein the at least one chemotherapeutic agent is dacarbazine.

8. The method of claim 6, wherein the at least one chemotherapeutic agent is docetaxel.

9. The method of claim 6, wherein the at least one chemotherapeutic agent is doxorubicin.

10. The method of claim 6, wherein the at least one chemotherapeutic agent is liposomal doxorubicin.

11. The method of claim 6, wherein the at least one chemotherapeutic agent is vincristine.
